(19) United States
(12) Patent Application Publication
(10) Pub. No.: US 2016/0016166 A1
(43) Pub. Date: Jan. 21, 2016

(54) MOLECULAR DIAGNOSTIC DEVICES WITH MAGNETIC COMPONENTS

(71) Applicant: DIAGNOSTICS FOR ALL, INC., Cambridge,, MA (US)

(72) Inventors: Jason Rolland, Belmont, MA (US); John Thomas Connelly, Somerville, MA (US)

(21) Appl. No.: 14/771,551

(22) PCT Filed: Mar. 14, 2014
(86) PCT No.: PCT/US2014/027894

§ 371 Date: Aug. 31, 2015

Related U.S. Application Data
(60) Provisional application No. 61/784,938, filed on Mar. 14, 2013.

(51) Int. Cl. B01L 3/00; C12Q 1/68

(52) U.S. Cl. CPC B01L 3/50273; C12Q 1/6844; B01L 2200/0605; B01L 2300/0636; B01L 2300/0887; B01L 2300/16; B01L 2300/126

(57) ABSTRACT

The invention provides disposable microfluidic devices that incorporate magnetic media and methods of using such devices to perform diagnostic assays on a sample. One exemplary microfluidic device comprises a first magnetic layer, a second magnetic layer, and a substantially planar member containing at least one hydrophilic region, where the substantially planar member is disposed between the first magnetic layer and the second magnetic layer, and the magnetic layers provide an attractive force useful for reducing the loss of fluid and/or s reagent from the device.

### CROSS REFERENCE TO RELATED APPLICATIONS

This application claims the benefit of and priority to U.S. Provisional Patent Application Ser. No. 61/784,938, filed Mar. 14, 2013, the contents of which are hereby incorporated by reference.

### GOVERNMENT SUPPORT

This invention was made with government support under Grant No. HR0011-12-2-0010 provided by the Defense Advanced Research Projects Agency. The government has certain rights in the invention

### FIELD OF THE INVENTION

The invention relates to devices for analyzing biological samples and their use. In particular, the invention provides point-of-care microfluidic devices that can perform diagnostic assays at low cost and with little accumulation of biohazardous waste. Incorporation of magnetic components into the devices described herein provides a solution for problems, such as fluid loss, associated with operation of certain microfluidic devices and allows for integration of magnetic reagents in the assays performed.

### BACKGROUND

Analytical devices for detecting the presence of biological materials are important for the detection and diagnosis of medical disorders. Cheap, disposable analytical devices capable of detecting biologically significant analytes are particularly important for providing basic medical testing to patient populations without ready access to a hospital or other medical facilities with instrumentation for analytical analysis of biological samples.

Microfluidic systems have attracted increasing interest due to their diverse and widespread potential applications. Lateral-flow microfluidic devices are two dimensional (2-D) and are used for applications where fluids need to be transported in a single plane, in series or in parallel. Three dimensional (3-D) microfluidic devices have been developed to accommodate more complex fluid flow schemes, allowing for controlled introduction and removal of multiple reaction components, execution of isolated reaction steps, and integration of multiple reactions and detection assays within the same device. The introduction of a sliding strip component into some 3-D devices allows for increased control and complexity with regard to the execution and type of assays performed. Using very small volumes of sample, microfluidic systems can carry out complicated biochemical reactions to acquire important chemical and biological information. Devices have been designed to detect patient genetic data and perform disease diagnostics, including assays of DNA, RNA, and protein identity and quantification. Among other advantages, microfluidic systems shorten the response time of reactions, reduce the required amount of samples and reagents, decrease the amount of biohazard waste generated, require little or no additional equipment for use, and have the potential to significantly reduce the cost associated with gathering the type of diagnostic information they provide.

3-D microfluidic devices are often in the form of multiple layered sheets of three or more. Reaction components can be introduced via a top layer, and the reaction takes place within the intermediate layers of the device. One challenge encountered with certain 3-D microfluidic devices, particularly those with a sliding layer, is undesirable loss of fluid by evaporation or other means. Therefore, there is a need for 3-D microfluidic devices constructed in a way that reduces the loss of fluid from the device. The present invention addresses this need and provides other related advantages.

### SUMMARY

The invention provides microfluidic devices comprising magnetic components and methods of using the devices to detect the presence of an analyte in a sample. The devices are low-cost, easy to use, require minute amounts of biological sample, generate relatively little biological waste, and generate valuable diagnostic data in a short period of time. Magnetic components of the device can help seal the device by providing a constant attractive force between layers, thereby reducing the loss of fluid and/or reagents used during operation of the device. For example, operation of some 3-D microfluidic devices requires a heating step that can result in the evaporation of reaction components if the device is insufficiently sealed. The seal created by attraction between magnetic members of the devices described herein helps to alleviate the loss of fluid and/or reagents during heated reactions, particularly if used in combination with a very thin layer of inert grease (e.g., a fluoropolymer grease). Additionally, the aforementioned seal created by attractive magnetic components can be useful in ensuring that gaps do not exist within the device that might allow reagents to leak out of interstitial space between layers of the device. In this respect, the seal provided by the magnetic components is also useful in devices that incorporate sliding components that may be more prone to loss of reagents from the reaction site during movement of the sliding member.

Accordingly, one aspect of the invention provides a three-dimensional device for processing biological samples. The device comprises (1) a first magnetic layer comprising magnetic media, a first inlet through which fluid can pass, and a reagent inlet through which fluid can pass; and (2) a second magnetic layer comprising (i) magnetic media configured to provide an attractive force between said first magnetic layer and said second magnetic layer, and (ii) a hydrophilic region; wherein the second magnetic layer is movable relative to the first magnetic layer to permit establishment of fluid flow communication serially between an inlet in the first magnetic layer and the hydrophilic region in the second magnetic layer. The attractive force between the first magnetic layer and the second magnetic layer holds the device together and helps prevent fluid and/or reagents from evaporating or otherwise leaking out of the device. Inlets in the first magnetic layer are desirably positioned so that serial movement of the second magnetic layer serially brings the hydrophilic region in the second magnetic layer into fluid communication with an inlet in the first magnetic layer. For example, the device may be configured so that applying a sample to the first inlet channels the sample to the hydrophilic region in the second magnetic layer, then the second magnetic layer is moved to bring the hydrophilic region into fluid communication with a different inlet in the first magnetic layer, such as a reagent inlet or a buffer wash inlet.

The device may comprise additional features, such as a track, additional magnetic layer(s), and/or one or more substantially planar members. The device may contain a track housing the second magnetic layer, in order to guide movement of the second magnetic layer. The track may contain substantially planar members along which the second magnetic layer may slide laterally. In some embodiments, the track may contain magnetic media configured to provide an attractive force between the first magnetic layer and the track. The device may also comprise additional magnetic layers, in order to provide additional attractive force holding the device together. In certain embodiments, the device comprises a third magnetic layer. The third magnetic layer is desirably located adjacent to the second magnetic layer, opposite the first magnetic layer. The third magnetic layer may contain an outlet through which fluid can pass. The device may also contain a first substantially planar member comprising a hydrophilic region in fluid communication with the hydrophilic region in the second magnetic layer, wherein said first substantially planar member is located adjacent to the second magnetic layer opposite the first magnetic layer. The first substantially planar member may be a hydrophilic material comprising a fluid-impermeable barrier that defines a boundary of a hydrophilic region. In certain embodiments, the first substantially planar member comprises paper, cloth, and/or a polymer film.

Another aspect of the invention provides a device comprising (1) a first magnetic layer comprising magnetic media and a sample inlet through which fluid can pass; (2) a second magnetic layer comprising magnetic media configured to provide an attractive force between said first magnetic layer and said second magnetic layer; and (3) a first substantially planar member comprising a fluid-impermeable barrier that defines a boundary of at least one hydrophilic region in fluid communication with said sample inlet, wherein said first substantially planar member is disposed between the first magnetic layer and the second magnetic layer. This arrangement allows for creation of a magnetic seal around the substantially planar member. In some embodiments, the device may include one or more additional substantially planar members, each of which may have a fluid-impermeable barrier that defines the boundary of at least one hydrophilic region to direct the flow of fluid through the substantially planar member. These additional planar members may be located adjacent to the first substantially planar member such that they either are situated or can be placed in fluid communication with the hydrophilic region of the first substantially planar member. Thus, in some embodiments at least one substantially planar member may be moveable relative to the other members. In some embodiments, the hydrophilic region in one of the substantially planar members may include assay reagents, especially an assay reagent capable of acting as a colorimetric indicator of analyte presence or amount. In some embodiments, the second magnetic layer may include a transparent region that allows a user to observe the colorimetric indicator. Another feature of devices with this configuration is that a user may pull apart layers of the device—by applying force sufficient to overcome the attraction between the magnetic layers—in order to obtain access to a diagnostic assay result from an assay performed in the interior of the device and not readily observable from the exterior of the device.

Another aspect of the invention provides a three-dimensional device for processing biological samples, wherein the device comprises: (1) a first magnetic layer comprising magnetic media and a sample inlet through which fluid can pass; (2) a first substantially planar member adjacent to the first magnetic layer and comprising a fluid-impermeable barrier that defines a boundary of at least one hydrophilic region that is in fluid communication with the sample inlet of the first magnetic layer; (3) a second magnetic layer comprising a region through which fluid can pass and magnetic media configured to provide an attractive force between said first magnetic layer and said second magnetic layer; said second magnetic layer being located adjacent to the first substantially planar member on the side of the first substantially planar member opposite said first magnetic layer, and said second magnetic layer being moveable relative to the first substantially planar member to permit establishment of fluid flow communication serially between a hydrophilic region of said first substantially planar member and the region of said second magnetic layer through which fluid can pass; (4) a second substantially planar member disposed on the side of the second magnetic layer opposite said first substantially planar member, said second substantially planar member comprising a fluid-impermeable barrier that defines a boundary of at least one hydrophilic region that can be in fluid communication with the region of the second magnetic layer through which fluid can pass depending on the position of the second magnetic layer; and (5) a third magnetic layer comprising magnetic media configured to provide an attractive force between said second magnetic layer and said third magnetic layer, wherein said third magnetic layer is disposed on the side of the second substantially planar member opposite the second magnetic layer. The second magnetic layer is moveable relative to the first substantially planar member, allowing establishment of fluid flow communication serially between at least one hydrophilic region of the first substantially planar member and the region of the second magnetic layer through which fluid can pass. The region of the second magnetic layer through which fluid passes optionally comprises a hydrophilic medium such as paper. The second substantially planar member may be in fluid communication with the region of the second magnetic layer through which fluid can pass, depending on the position of the second magnetic layer, and the second substantially planar member may include an assay reagent capable of providing a colorimetric readout of analyte presence or amount. The third magnetic layer may include a transparent region that allows a user to view the assay reagent providing the colorimetric readout. This configuration provides a superior seal due to the three layers comprising magnetic media—such a seal is particularly beneficial to prevent undesirable loss of fluid and/or reagents that can occur in devices with sliding components but that lack the magnetic layers. Maintaining a seal after a nucleic acid amplification reaction is of particular importance given the proclivity of such amplified material to pose a contamination threat to the lab environment.

Some embodiments of the device above may include a third substantially planar member disposed between the second magnetic layer and the second substantially planar member, wherein the third substantially planar member has a fluid-impermeable barrier that defines a boundary of at least one hydrophilic region that is always in fluid communication with a region of the second magnetic layer through which fluid can pass.

Some embodiments of the device above may incorporate a track along which the second magnetic layer may slide laterally. The track may itself comprise magnetic media such that it provides an attractive force between itself and both the first and third magnetic layer. In some embodiments of the device, both the track and the second magnetic layer may be made of double-side magnetic sheets.

Another aspect of the invention provides a three-dimensional device for processing biological samples, wherein the device comprises: (1) a first substantially planar member comprising a fluid-impermeable barrier that defines a boundary of at least one hydrophilic region; and (2) a magnetic layer comprising magnetic media, said magnetic layer being located adjacent to and in fluid communication with a hydrophilic region of the first substantially planar member, and the magnetic layer being moveable relative to the first substantially planar member. Some embodiments of the device may incorporate a second substantially planar member containing a fluid-impermeable barrier that defines the boundary of at least one hydrophilic region. This second substantially planar member may be situated adjacent to the magnetic layer on the side opposite the first substantially planar member. This configuration of the device is contemplated to be useful for manipulating magnetic reagents (e.g., Dynabeads®) that can be employed in biological assays.

Another aspect of the invention provides a three-dimensional, microfluidic assay device for detection of analytes by applying a fluid sample onto at least one substantially planar member comprising a hydrophilic region containing one or more test zones defined by a fluid-impermeable barrier, wherein the improvement to the device comprises a first magnetic layer comprising magnetic media and a sample inlet through which fluid can pass to a hydrophilic region of at least one substantially planar member of the device containing one or more test zones, and a second magnetic layer comprising magnetic media configured to provide an attractive force between said first magnetic layer and said second magnetic layer, wherein at least one substantially planar member comprising a hydrophilic region containing one or more test zones defined by a fluid-impermeable barrier is disposed between the first magnetic layer and the second magnetic layer.

The invention also provides a method for using devices described herein in the detection of an analyte in a sample. The method comprises adding a sample to the device, and detecting the presence of an analyte in said sample.

### BRIEF DESCRIPTION OF FIGURES

FIG. 1 depicts an exemplary 3-D microfluidic device incorporating magnetic components including stationary and sliding magnetic film layers that act in tandem with substantially planar members comprising fluid impermeable barriers with defined hydrophilic regions to perform diagnostic assays on a sample.

FIG. 2 depicts illustrations of exemplary 3-D microfluidic devices incorporating magnetic components.

FIG. 3 depicts an exemplary 3-D microfluidic device incorporating magnetic components.

FIG. 4 is a bar graph showing results of water-loss analysis from a microfluidic device containing magnetic components, as described in Example 1.

FIG. 5 depicts results of a LAMP reaction, as described in Example 2.

FIG. 6 is a bar graph showing results of an assay to determine the analytical sensitivity of a LAMP reaction performed in an assay device, as described in Example 2.

FIG. 7 illustrates procedures for performing an analytical test using a device described herein, wherein Step 1 is to apply sample to the reaction disc; Step 2 is to slide the strip to move the reaction disc to a wash port and apply wash buffer to reaction disc; Step 3 is to slide the strip to move the reaction disc to a reagent port and apply reagent mix (e.g., Master Mix) to the reaction disc; Step 4 is to slide the strip to a sealed amplification zone and incubate the device (e.g., heat the device to elevated temperature); and Step 5 is to slide the strip to move the reaction disc to a detection window and apply a detection reagent (e.g., SYBR Green I followed by exposure to ultra-violet radiation) and visualize the results (e.g., by taking a picture using a camera phone), as described in Example 2.

FIG. 8 is a bar graph showing results of a LAMP reaction challenged with whole, live E. coli cells, as described in Example 2.

FIG. 9 is a bar graph showing the impact of increased sample volume on the number of positive and negative results, as further described in Example 2. Samples of 0.05 cells/μL E. coli in human plasma were applied in 10 μL increments to reactions discs in sliding strip devices and processed through the integrated sample preparation, amplification and detection procedures of the device (n=8). Signals from all replicates of 10 μL and 20 μL (˜0.5 and 1 cell applied to discs) samples remained below the limit of detection (LOD), determined as the mean plus 3 times the standard deviation of a NTC run in parallel, and were thus scored as negative. With 40 μL and 60 μL samples (˜2 and 3 cells, respectively), the number of replicates scoring positive, above the LOD, increased to 7 and 8, respectively.

FIG. 10 is an exploded view of a microfluidic device showing three magnetic layers and multiple laminate layers, in which the bottom (i.e., third) magnetic layer contains a region for lateral flow of fluid.

FIG. 11 is a condensed view of the microfluidic device from FIG. 10 in which laminate layers (e.g., a substantially planar, hydrophilic substrates) are bonded to the appropriate magnetic layer.

### DETAILED DESCRIPTION

The invention provides microfluidic devices comprising magnetic components and methods of using the devices to detect the presence of an analyte in a sample. The devices are low-cost, easy to use, require minute amounts of biological sample, generate relatively little biological waste, and can generate valuable diagnostic data in a short period of time. Magnetic components of the device help seal the device, thereby reducing the loss of fluid and/or reagents used during operation of the device. Features of the devices and methods of using the devices are described in sections below. The sections are arranged for convenience and information in one section is not limited to that section, but may be applied to other sections.

### Basic Device Configurations

### First Configuration

A first configuration of the device is a three-dimensional microfluidic device comprising: (1) a first magnetic layer comprising magnetic media, a first inlet through which fluid can pass, and a reagent inlet through which fluid can pass; and (2) a second magnetic layer comprising (i) magnetic media configured to provide an attractive force between said first magnetic layer and said second magnetic layer, and (ii) a hydrophilic region; wherein the second magnetic layer is movable relative to the first magnetic layer to permit establishment of fluid flow communication serially between an inlet in the first magnetic layer and the hydrophilic region in the second magnetic layer. The attractive force between the first magnetic layer and the second magnetic layer holds the device together and helps prevent fluid and/or reagents from evaporating or otherwise leaking out of the device

The device may optionally further comprise a first substantially planar member comprising a hydrophilic region in fluid communication with the hydrophilic region in the second magnetic layer, wherein said first substantially planar member is located adjacent to the second magnetic layer opposite the first magnetic layer. In certain embodiments, the first substantially planar member is a hydrophilic material comprising a fluid-impermeable barrier that defines a boundary of a hydrophilic region. In certain embodiments, the first substantially planar member comprises a material selected from the group consisting of paper, cloth, and polymer film. In certain other embodiments, the first substantially planar member comprises paper. The fluid-impermeable barriers may comprise a wax, poly(methylmethacrylate), an acrylate polymer, polystyrene, polyethylene, polyvinylchloride, a fluoropolymer, a photoresist, or a photo-polymerizable polymer that forms a hydrophobic polymer. The fluid-impermeable barriers that define boundaries of said plural hydrophilic regions may be produced by screening, stamping, printing or photolithography.

The device may optionally further comprise a third magnetic layer. In certain embodiments, the device comprises a third magnetic layer comprising (i) magnetic media configured to provide an attractive force between said second magnetic layer and said third magnetic layer, and (ii) an outlet through which fluid can pass, the third magnetic layer being located adjacent to the second magnetic layer opposite the first magnetic layer. In other embodiments, the device further comprises a third magnetic layer comprising (i) magnetic media configured to provide an attractive force between said second magnetic layer and said third magnetic layer, and (ii) an outlet through which fluid can pass, the third magnetic layer being located adjacent to the first substantially planar member opposite the second magnetic layer. Because certain diagnostic assays provide a colorimetric result, in certain the embodiments the third magnetic layer comprises a transparent region for viewing colorimetric indication of the amount of analyte present in a sample in the second magnetic layer.

The device may optionally further comprise a second substantially planar member comprising a hydrophilic region in fluid communication with an outlet in the third magnetic layer, wherein said second substantially planar member is located adjacent to the third magnetic layer opposite the second magnetic layer. In certain embodiments, the second substantially planar member comprises a material selected from the group consisting of paper, cloth, and polymer film. In certain other embodiments, second substantially planar member comprises paper. In certain embodiments, the second substantially planar member is a hydrophilic material comprising a fluid-impermeable barrier that defines a boundary of a hydrophilic region. The fluid-impermeable barriers may comprise a wax, poly(methylmethacrylate), an acrylate polymer, polystyrene, polyethylene, polyvinylchloride, a fluoropolymer, a photoresist, or a photo-polymerizable polymer that forms a hydrophobic polymer. The fluid-impermeable barriers that define boundaries of said plural hydrophilic regions may be produced by screening, stamping, printing or photolithography.

The device may optionally further comprise a track housing the second magnetic layer. The track may comprise substantially planar members along which the second magnetic layer may slide laterally. In certain embodiments, the track comprises magnetic media configured to provide an attractive force between the first magnetic layer and the track. In certain other embodiments, the track and second magnetic layer both comprise a double-sided magnetic sheet.

The first inlet can be for receiving a sample, i.e., a sample inlet. A sample containing an analyte is applied to the first inlet, and the sample is channeled to the hydrophilic region in the second magnetic layer.

The device may optionally further comprise additional inlets. In certain embodiments, the first magnetic layer further comprises an inlet for receiving a wash buffer, wherein establishment of fluid communication between said inlet for receiving a wash buffer and said hydrophilic region in said second magnetic layer is effected by movement of said second magnetic layer relative to the first magnetic layer. In certain other embodiments, the first magnetic layer further comprises an inlet for receiving an analyte amplification reagent, wherein establishment of fluid communication between said inlet for receiving an analyte amplification reagent and said hydrophilic region in said second magnetic layer is effected by movement of said second magnetic layer relative to the first magnetic layer.

The reagent input can be used to introduce a single type of reagent or multiple reagents. In certain embodiments, the reagent inlet is for receiving an analyte detection reagent, wherein establishment of fluid communication between said inlet for receiving an analyte detection reagent and said hydrophilic region in said second magnetic layer is effected by movement of said second magnetic layer relative to the first magnetic layer. In certain embodiments, the analyte detection reagent provides a colorimetric indication of the presence of an analyte in the sample.

The device may optionally comprise an assay reagent. For example, in certain embodiments, the assay reagent is located in one of the layers of the device in a location that comes into fluid contact with the sample during operation of the device. In certain embodiments, the first magnetic layer may contain an inlet, wherein the inlet itself is a porous material comprising an assay reagent (e.g., such as for analyte detection). Administering fluid to such an inlet can transport the reagent into the hydrophilic region in the second magnetic layer when the second magnetic layer has been moved into fluid communication with such inlet in the first magnetic layer. Alternatively, the assay reagent is deposed in a reagent zone in the second magnetic layer, where said reagent zone comes into fluid communication with the sample during operation of the device. The assay reagent desirably provides a colorimetric indication of the amount of analyte present in a sample. Because certain diagnostic assays provide a colorimetric result, in certain embodiments the third magnetic layer comprises a transparent region for viewing colorimetric indication of the amount of analyte present in a sample. Exemplary assay reagents are described in more detail below.

The device may optionally further comprise a site bounded by a seal for inhibiting evaporation of fluid from the device. The seal may comprise a grease. Grease applied to the periphery, portions, or throughout one or more layers of the device can help reduce loss of fluid or reagents from the device. The device can be characterized according to the amount of water loss. For example, in certain embodiments, the device is characterized by less than 1% w/w of water contained in the site bounded by a seal evaporates when the device is heated to 65° C. for a duration of 1 hour.

The device may optionally further comprise a positive control zone and/or a negative control zone. In certain embodiments, the first magnetic layer contains an inlet through which fluid can pass that leads to a positive control zone in the second magnetic layer. In certain other embodiments, the first magnetic layer contains an inlet through which fluid can pass that leads to a negative control zone in the second magnetic layer.

The hydrophilic region in the second magnetic layer can be further characterized according to the composition of the hydrophilic region. In certain embodiments, the hydrophilic region in the second magnetic layer comprises paper. In certain other embodiments, the hydrophilic region in the second magnetic layer comprises paper, cloth, or a polymer film. In certain embodiments, the hydrophilic region in the second magnetic layer comprises a reagent that causes a cell to lyse. In certain other embodiments, the hydrophilic region in the second magnetic layer comprises one or more of uric acid or a salt thereof, a detergent, a base, and a chelating agent. In certain other embodiments, the hydrophilic region in the second magnetic layer comprises uric acid or a salt thereof, a base, and a chelating agent. In certain other embodiments, the hydrophilic region in the second magnetic layer comprises a detergent, such as an alkali metal alkyl sulfate, such as sodium dodecyl sulfate. In yet other embodiments, the hydrophilic region in the second magnetic layer comprises a chaotropic agent, such as guanidinium thiocyanate.

The device may optionally further comprise a filter. In certain embodiments, a filter is in fluid communication with the sample inlet in the first magnetic layer. In certain embodiments, the sample inlet in the first magnetic layer comprises a filter, such as paper.

The device may be further characterized according to the composition of magnetic media in the device. In certain embodiments, the magnetic media comprises ferrite. In certain other embodiments, the magnetic media comprises a mixture of ferrite and a binder selected from the group consisting of synthetic vinyl rubber, poly(dimethylsiloxane), a polyurethane, a natural rubber, a fluoroelastomer, and combinations thereof. In certain other embodiments, the magnetic media comprises a mixture of ferrite and synthetic vinyl rubber.

### Second Configuration

A second configuration of the device is a three-dimensional microfluidic device comprising: (1) a first magnetic layer comprising magnetic media and a sample inlet through which fluid can pass; (2) a second magnetic layer comprising magnetic media configured to provide an attractive force between said first magnetic layer and said second magnetic layer; and (3) a first substantially planar member comprising a fluid-impermeable barrier that defines a boundary of at least one hydrophilic region in fluid communication with said sample inlet, wherein said first substantially planar member is disposed between the first magnetic layer and the second magnetic layer. The attractive force between the first magnetic layer and the second magnetic layer holds the device together and helps prevent fluid and/or reagents from evaporating or otherwise leaking out of the device.

The device may optionally further comprise a second substantially planar member comprising a fluid-impermeable barrier that defines a boundary of at least one hydrophilic region, wherein said second substantially planar member is located adjacent to the first substantially planar member. In certain embodiments, the first substantially planar member and the second substantially planar member are moveable relative to each other to permit establishment of fluid flow communication serially between a hydrophilic region of said first substantially planar member and a hydrophilic region of said second substantially planar member.

The device may optionally comprise a fluid-impermeable layer comprising one or more openings to permit fluid flow. The fluid-impermeable layer may be disposed adjacent to a substantially planar member.

The device may optionally comprise an assay reagent. For example, in certain embodiments, a hydrophilic region in the second substantially planar member comprises an assay reagent. The assay reagent desirably provides a colorimetric indication of the amount of analyte present in a sample. Because certain diagnostic assays provide a colorimetric result, in certain the embodiments the second magnetic layer comprises a transparent region for viewing colorimetric indication of the amount of analyte present in a sample.

### Third Configuration

A third configuration of the device is a three-dimensional microfluidic device comprising (1) a first magnetic layer comprising magnetic media and a sample inlet through which fluid can pass; (2) a first substantially planar member adjacent to the first magnetic layer and comprising a fluid-impermeable barrier that defines a boundary of at least one hydrophilic region that is in fluid communication with the sample inlet of the first magnetic layer; (3) a second magnetic layer comprising a region through which fluid can pass and magnetic media configured to provide an attractive force between said first magnetic layer and said second magnetic layer; said second magnetic layer being located adjacent to the first substantially planar member on the side of the first substantially planar member opposite said first magnetic layer, and said second magnetic layer being moveable relative to the first substantially planar member to permit establishment of fluid flow communication serially between a hydrophilic region of said first substantially planar member and the region of said second magnetic layer through which fluid can pass; (4) a second substantially planar member disposed on the side of the second magnetic layer opposite said first substantially planar member, said second substantially planar member comprising a fluid-impermeable barrier that defines a boundary of at least one hydrophilic region that can be in fluid communication with the region of the second magnetic layer through which fluid can pass depending on the position of the second magnetic layer; and (5) a third magnetic layer comprising magnetic media configured to provide an attractive force between said second magnetic layer and said third magnetic layer, wherein said third magnetic layer is disposed on the side of the second substantially planar member opposite the second magnetic layer. The attractive force between the magnetic layers holds the device together and helps prevent fluid and/or reagents from evaporating or otherwise leaking out of the device.

The device may optionally further comprise a third substantially planar member disposed between the second magnetic layer and the second substantially planar member, wherein the third substantially planar member comprises a fluid-impermeable barrier that defines a boundary of at least one hydrophilic region that is always in fluid communication with a region of the second magnetic layer through which fluid can pass.

The device may optionally further comprise a track housing the second magnetic layer, the track comprising substantially planar members along which the second magnetic layer may slide laterally. In certain embodiments, the track comprises magnetic media configured to provide an attractive force between the first magnetic layer and the track, and an attractive force between the third magnetic layer and the track. In certain embodiments, the track and second magnetic layer both comprise a double-sided magnetic sheet.

As described above, second magnetic layer comprises a region through which fluid can pass. This region may be an open space or may be a composition through which fluid can pass. In certain embodiments, the region through which fluid can pass in the second magnetic layer comprises a hydrophilic medium (such as paper, cloth, or a porous polymer layer). In certain other embodiments, the region through which fluid can pass in the second magnetic layer comprises paper.

The device may optionally comprise an assay reagent. For example, in certain embodiments, a hydrophilic region in the second substantially planar member comprises an assay reagent. The assay reagent desirably provides a colorimetric indication of the amount of analyte present in a sample. Because certain diagnostic assays provide a colorimetric result, in certain embodiments the third magnetic layer comprises a transparent region for viewing colorimetric indication of the amount of analyte present in a sample. Exemplary assay reagents are described in more detail below.

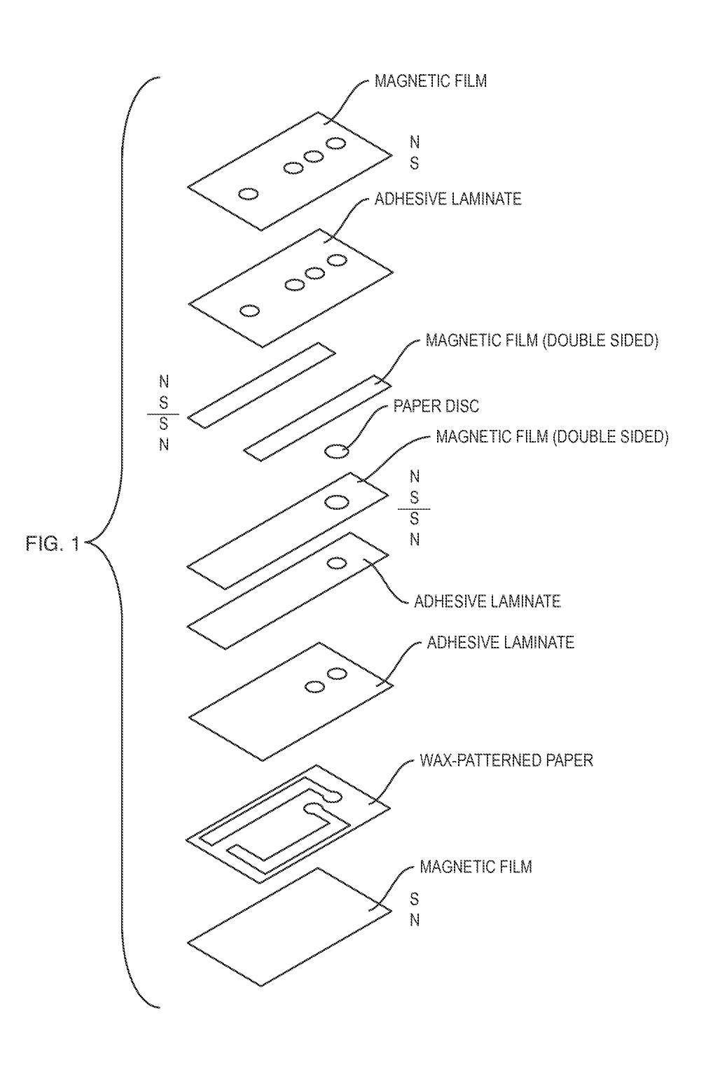

A more specific example of a device having features of the third configuration generally described above is presented in FIG. 1. The device in FIG. 1 has a sliding member comprising a double-sided magnetic sheet, a paper reaction disc, and a laminate film layer. The sliding strip containing the paper disc is attracted to a top magnetic piece as well as a bottom magnetic layer. The device in FIG. 1 is designed such that the strip is slid from station to station where optionally a different fluidic operation can be performed on the paper disc in each station. For example, a blood sample may be introduced to the first port where lysis and capture occurs on the paper disc. It is then slid to a second port where a wash buffer is introduced to purify the captured target. The disc is then slid to a third station where it receives amplification reagents (e.g., for performing loop-mediated isothermal amplification or other amplification reactions). Next, the disc is slid to a hermetically-sealed amplification region where heat is applied to drive the reaction. Finally, the disc is slid to a fifth station where it receives reagents necessary for detection. The magnetic attraction ensures a proper seal between the sliding strip and the layers above and below the strip. This is particularly important during the amplification step where maintaining a hermetic seal completely surrounding the paper reactor is important. The magnetic sheeting can be used in combination with thin layers of inert greases such as Krytox® available form DuPont to enhance sealing of the device.

### Fourth Configuration

A fourth configuration of the device is a three-dimensional microfluidic device comprising: (1) a first substantially planar member comprising a fluid-impermeable barrier that defines a boundary of at least one hydrophilic region; and (2) a magnetic layer comprising magnetic media, said magnetic layer being located adjacent to and in fluid communication with a hydrophilic region of the first substantially planar member, and the magnetic layer being moveable relative to the first substantially planar member. This configuration is contemplated to be particularly useful to manipulate analytes that are attached to magnetic beads. Movement of the magnetic layer of the device can be used to transport the magnetic beads to different locations of the device, where different chemical manipulations may be performed at different locations in the device. It is contemplated that magnetic beads (e.g., Dynabeads®) could be used to capture a target analyte, and then the conjugate formed by the magnetic bead/target analyte could be transported to different regions of the device using magnetic force. A particular example is the mixing of a blood sample with magnetic beads to isolate an analyte from the blood sample, and the resulting conjugate of analyte/magnetic beads is carried to particular stations within the magnetic device using a magnetic sliding strip.

In certain embodiments, the device further comprises a second substantially planar member comprising a fluid-impermeable barrier that defines a boundary of at least one hydrophilic region; said second substantially planar member being located adjacent to the magnetic layer on the side opposite the first substantially planar member.

In certain embodiments, the magnetic layer comprises a fluid-impermeable barrier that defines a boundary of at least one hydrophilic region that can be in fluid communication with a hydrophilic region of said first substantially planar member.

### Fifth Configuration

A fifth configuration of the device is a three-dimensional, microfluidic assay device for detection of analytes by applying a fluid sample onto at least one substantially planar member comprising a hydrophilic region containing one or more test zones defined by a fluid-impermeable barrier, the improvement comprising a first magnetic layer comprising magnetic media and a sample inlet through which fluid can pass to a hydrophilic region of at least one substantially planar member of the device containing one or more test zones, and a second magnetic layer comprising magnetic media configured to provide an attractive force between said first magnetic layer and said second magnetic layer, wherein at least one substantially planar member comprising a hydrophilic region containing one or more test zones defined by a fluid-impermeable barrier is disposed between the first magnetic layer and the second magnetic layer.

Various features of the devices described above are further described in the sections below:

### Magnetic Layers

Devices described herein incorporate magnetic media in a magnetic layer. In certain embodiments, the magnetic media comprises ferrite. In certain other embodiments, the magnetic media comprises a mixture of ferrite and a binder selected from synthetic vinyl rubber, poly(dimethylsiloxane), a polyurethane, a natural rubber, a fluoroelastomer, and combinations thereof. In certain other embodiments, the magnetic media comprises a mixture of ferrite and synthetic vinyl rubber. In certain embodiments, the magnetic media are magnetic strips in the form of planar members. The attractive force generated by the magnetic media should be sufficient to hold the device together and desirably reduce fluid loss from the device. In some embodiments, a magnetic layer will be made of double-sided magnetic strips with similar poles aligned on the inner face of the magnetic layer and the magnetic strips will be joined by a suitable medium such as epoxy or a pressure-sensitive adhesive. It is contemplated that magnetic layers made of double-sided magnetic strips will be especially useful for a device that consists of three or more magnetic layers, where an internal magnetic layer can be made of double-sided magnetic strips. In some embodiments, an internal sliding member and/or a track for the sliding member may be made of double-sided magnetic strips. It is appreciated that a set of sequential magnetic layers meant to provide an attractive force should be situated in the device such that their opposing poles face one another when situated in the device, unless the sequential magnetic layers consist of double-sided magnetic strips held together adhesively by an appropriate media such as epoxy or a pressure-sensitive adhesive.

### Substantially Planar Members

Substantially planar members of the device can be hydrophilic materials. The hydrophilic materials can be manipulated to provide isolated hydrophilic regions defined by a fluid-impermeable barrier added to the hydrophilic material. The substantially planar member can be made from any hydrophilic material that wicks fluid by capillary action. Exemplary hydrophilic materials include chromatographic paper, filter paper, cellulosic paper, paper towels, toilet paper, tissue paper, notebook paper, Kim Wipes, VWR Light-Duty Tissue Wipers, Technicloth Wipers, newspaper, cloth, or a polymer film such as nitrocellulose and cellulose acetate. In certain embodiments, the substantially planar members comprise a sheet of paper, nitrocellulose, cellulose acetate, cloth, or a porous polymer film. In certain other embodiments, the substantially planar members comprise paper, such as Whatman chromatography filter paper No. 1.

In certain embodiments, the first substantially planar member comprises a material selected from the group consisting of paper, cloth, and polymer film. In yet other embodiments, the first substantially planar member comprises paper. In certain embodiments, the second substantially planar member comprises a material selected from the group consisting of paper, cloth, and polymer film. In yet other embodiments, the second substantially planar member comprises paper.

The fluid-impermeable barriers direct fluid through the device. The fluid-impermeable barriers substantially permeate the thickness of the layer to define plural flow paths. The fluid-impermeable barriers may be produced by screening, stamping, printing, or photolithography. The fluid-impermeable barriers may comprise a wax, poly(methylmethacrylate), a photoresist, an acrylate polymer, a fluoropolymer, polystyrene, polyethylene, polyvinylchloride, or a photo-polymerizable monomer that forms a hydrophobic polymer. Procedures for imparting patterns on hydrophilic materials, along with microfluidic devices formed from patterned paper, are described in WO 2008/048083, WO 2009/121043, WO 2009/121041, WO 2009/121037, WO 2010/102294, WO 2010/102279, and WO 2011/097412, each of which are incorporated by reference for all purposes.

When using photolithography to install the fluid-impermeable barrier, the hydrophilic material is first soaked in photoresist, and then photolithography is used to pattern the photoresist to form fluid impervious barriers following the procedures described in, e.g., International Patent Application WO 2009/121037. Photoresist materials used for patterning porous, hydrophilic material may include SU-8 photoresist, SC photoresist (Fuji Film), poly(methylmethacrylate), acrylates, polystyrene, polyethylene, polyvinylchloride, and any photopolymerizable monomer that forms a hydrophobic polymer.

When using micro-contact printing or wax printing to install the fluid-impermeable barrier, a polymer or wax is applied in a defined pattern to the hydrophilic layer. For example, when using micro-contact printing to install the fluid-impermeable barrier, a “stamp” of defined pattern is “inked” with a polymer, and pressed onto and through the hydrophilic medium such that the polymer soaks through the medium; thus, forming barriers of that defined pattern. When using wax printing to install the fluid-impermeable barrier, the wax material may be hand-drawn, printed, or stamped onto a hydrophilic substrate. In embodiments where the wax material is a solid ink or a phase change ink, the ink can be disposed onto paper using a paper printer. Particular printers that can use solid inks or phase change inks are known in the art and are commercially available. One exemplary printer is a Phaser™ printer (Xerox Corporation). In such embodiments, the printer disposes the wax material onto paper by initially heating and melting the solid ink to print a preselected pattern onto the paper. The printed paper may be subsequently heated, e.g., by baking the paper in an oven, to melt the wax material (solid ink) to form hydrophobic barriers.

The wax material can be disposed onto a hydrophilic substrate in any predetermined pattern, and the feature sizes can be determined by the pattern and/or the thickness of the substrate. For example, a device can be produced by printing wax lines onto paper (e.g., chromatography paper) using a solid ink printer. The dimensions of the wax lines can be determined by the feature sizes of the device and/or the thickness of the paper. For example, the wax material can be printed onto paper at a line thickness of about 100 μm, about 200 μm, about 300 μm, about 400 μm, about 500 μm, about 600 μm, about 700 μm, about 800 μm, about 900 μm, about 1 mm, or thicker. The thickness of the wax to be printed can be determined by, e.g., analyzing the extent to which the wax permeates through the thickness of the substrate after heating. The wax material may be patterned on one or both sides of the hydrophilic material. Additional information on wax printing can be found in, for example, international patent application WO 2010/102294, which is hereby incorporated by reference.

Additional description of materials that can be used in the substantially planar member and the construction of three-dimensional assay devices is described in, for example, WO 2009/121037, which is hereby incorporated by reference.

### Site Bounded by a Seal

Magnetic layers in the device help to seal the device, thereby reducing the loss of fluid or reagents from the device. In addition, the device may comprise a site bounded by a seal for inhibiting evaporation of fluid from the device. The seal may comprise a grease. Grease applied to the periphery, portions, or throughout one or more layers of the device can help reduce loss of fluid or reagents from the device.

In devices with a sliding strip, the device may be configured with a seal to prevent drying, evaporation, or loss of the sample or assay reagents due to movement of a test zone or other region of the device containing fluid and/or assay reagents. For example, in certain embodiments, the device can be configured with a hermetic seal between the layers of the device by depositing a layer of grease or oil. Creation of an evaporation-resistant seal can be achieved by incorporation of grease into the device, such that a layer of grease is placed between individual layers of the device, or, for example, on the top surface of a sliding strip member exclusive of the area comprising the sample, such as the area comprising the reaction disc. In a preferred embodiment, Krytox® fluorinated polymer grease is used to create such a seal. Additionally, the incorporation of magnetic planar members surrounding internal layers of the device and aligned with their opposite poles facing one another provides an attractive force suitable to independently create and/or reinforce the strength of any seal provided by other reagents disposed in the device for the purpose of creating a fluid-impermeable seal.

### Inlet for Receiving Reagents

Devices may contain reagents in one or more areas in the device for detecting an analyte and/or the device may contain an inlet for receiving a reagent for detecting an analyte. The device described above in the first configuration comprise an inlet for a reagent. Other configurations may optionally further comprise, but do not require, an inlet for a reagent.

The device desirably comprises an inlet for receiving reagents useful for determining the presence or amount of an analyte in a sample. Various configurations of the device having an inlet for receiving a reagent are possible. In certain embodiments of the second device configuration described above, the first magnetic layer further comprises an inlet for receiving an analyte amplification reagent, said inlet being in fluid communication with a hydrophilic region specific for an analyte amplification reagent in the first substantially planar member. In certain other embodiments of the second device configuration described above, the first magnetic layer further comprises an inlet for receiving a wash buffer, said inlet being in fluid communication with a hydrophilic region specific for a wash buffer in the first substantially planar member. In yet other embodiments of the second device configuration described above, the first magnetic layer further comprises an inlet for receiving an analyte detection reagent, said inlet being in fluid communication with a hydrophilic region specific for an analyte detection reagent in the first substantially planar member. Analogous features may be present in other device configurations.

### Filter

The device may further comprise a filter. The filter may be located in the fluid flow path immediately after the location in which a clinical sample is applied to the device. The filter material may be selected in order to retain certain types of biological materials.

In certain embodiments, a filter is present in the sample inlet in the first magnetic layer of the device. In other embodiments, a filter is present in the device at a location where it is in fluid communication with a hydrophilic region to remove red blood cells from a blood sample. There is considerable experience and literature concerning plasma separation membranes, which effectively isolate plasma and allow it to wick into detection zones that contain chemistry to detect solutes disposed therein. Membranes such as Vivid GX plasma separation membrane available from Pall® corporation are highly effective. In other embodiments, the membrane can be a glass fiber membrane, or even a paper filter. In other embodiments, anti-blood cell antibodies may be attached to the membrane to facilitate capture of cells. In further embodiments, “scrubbing agents” may be added to the filter membrane or paper channels that are capable of capturing substances that may interfere with the reaction chemistry.

In some embodiments of the invention, it may be advantageous to lyse and filter components of microorganisms obtained in patient samples. A number of chaotropic reagents exist that can be used to lyse cells, viruses, and bacteria. A common chaotropic reagent is urea, which could be dried onto a paper zone and act as a lysing agent once in contact with a sample. Alternatively, paper products such as FTA® cards available from Whatman® contain proprietary agents embedded within the paper to lyse membranes and denature viral coat proteins (e.g., see: Whatman® Product Insert: “DNA Extraction from FTA® Cards Using the GenSolve DNA Recovery Kit.”; Bearinger, et al., IEEE transactions on Biomedical Engineering, 2010, 58, 805-808).

### Sliding Layer

Certain devices are configured to have a sliding layer. The sliding layer can be used to first receive test sample(s), then the sliding layer is moved to a second position to bring sample(s) into contact with certain buffer wash zones or a diagnostic agent for detecting the presence of particular molecules in the test sample. For example in one embodiment, the device is operated by applying a sample to the device via an inlet. The sample passes through a filter that separates unwanted components while allowing the desired portion of the sample to reach a test zone, located within a sliding member, to which desired sample components adhere. The test zone, comprising a hydrophilic region (e.g., a Whatman filter) can be further cleared of undesired sample components by means of moving the sliding member laterally within the plane of the device such that the test zone is brought into fluid communication with a second inlet of the device, through which a wash buffer can be applied to the filter.

In certain embodiments, said movement of the sliding member will also bring the test zone region into fluid communication with a second substantially planar member located below the first substantially member comprising the sliding member and test zone, said second substantially planar member comprising patterned channels suitable to direct excess wash buffer carrying undesired sample components away from the test zone. Further lateral movement of the sliding member in the same direction may bring the test zone into contact with other regions of the device suitable for further sample processing and product detection.

The sliding member can also be in the form of a disk that rotates. Rotating the disk brings the test zone into fluid communication with additional inlets (e.g., a second inlet of the device through which a wash buffer can be applied). Further rotating the disk in the same direction may bring the test zone into contact with other regions of the device suitable for further sample processing and product detection.

Additional aspects of devices containing a sliding strip layer are described in, for example, WO 2011/097412, which is hereby incorporated by reference.

### Nucleotide Amplification

In some embodiments of the invention, processing of biological samples will include amplification of isolated oligonucleotides. In a particular embodiment, DNA amplification will be carried out in a device using loop-mediated isothermal amplification (LAMP), described in Bearinger, et al. in IEEE transactions on Biomedical Engineering, 2010, 58, 805-808; Asiello and Baeumner in Lab on a Chip, 2011, 11, 1420; and Weigl et al. in Proc. of SPIE, 2008, 6886, 688604. In one embodiment, the device can be heated at 65° C. for 1 hour to facilitate oligonucleotide amplification by LAMP. Optionally, this can be achieved by placing the entire device in an oven or other suitable device set to the appropriate temperature. Following this isothermal amplification step, the sliding member comprising the test zone can be removed from the device and dried at 65° C. for 5 minutes before addition of a detection reagent.

An important consideration for applying any isothermal technique to a paper device is that all of these methods require heat to drive the amplification reaction. This step can be done simply by placing the device in an oven set at an appropriate temperature. However there are several ways to incorporate a heating element into a device. One method is to pattern an electric resistor into the portion of the device where heating is required. The Whitesides lab has used this technique to create valves and concentrators on paper microfluidic devices. See, for example, WO 2009/121041. The resistor can be operated using a “button battery” at a cost less than $0.10.

Another integrated heating option is the use of exothermic chemical reactions such as those found in “meals ready to eat” and commercial hand warming products. Weigl, et al., supra, have shown that by coupling these types of reactions to carefully chosen phase-change materials (such as waxes or metal alloys) it is possible to maintain constant elevated temperatures, well within the range needed for isothermal amplification, up to several hours. These techniques may be adapted to paper microfluidic devices to provide region-specific, sustained heating to isothermal amplification chemistries.

### Detection Zone for Detecting the Presence or Concentration of Biological Molecules

The device may optionally further comprise a detection zone for detecting the presence and/or concentration of a biological molecule. This feature yields a device that provides immediate diagnostic information for certain disease states or other diagnostic assays. Accordingly, in certain embodiments, the device further comprises, disposed in fluid communication with one or more of the flow paths within the device, a reagent for the detection of the presence or concentration of an analyte in a clinical sample in a detection zone.

The detection zone contains an assay reagent for detecting the presence and/or amount of a substance from the clinical sample, wherein the presence and/or amount of the substance is indicative of disease or health status or genetic makeup of the patient from which the clinical sample was obtained. Exemplary assay reagents include a nucleotide assay reagent, a protein assay reagent, an immunoassay reagent, glucose assay reagent, a sodium acetoacetate assay reagent, a sodium nitrite assay reagent, or a combination thereof.

Suitable detection schemes include, but are not limited to, the use of magnesium dyes (e.g., Xylenol orange, eriochrome black T) for the detection of free magnesium in LAMP DNA amplification reactions, reagents that fluoresce at specific wavelengths following DNA intercalation (e.g., propidium iodide, SYTO® Green (Invitrogen), and SYBR®-Green (Applied Biosystems)), electrochemical detection of amplified nucleic acids, such as described by Lu, et al. in Anal. Chem., 2012, 84 (4), pp 1975-1980, detection of amplified oligonucleotides by nucleic acid lateral flow (NALF) methods, and detection of protein or nucleotide analytes by enzyme-linked antibodies or antibody-coated colored particles. In some embodiments, the antibody-coated particle is selected from, but not limited to, the following: a colored polymer latex particle, a colloidal gold particle, a graphite particle, a quantum dot, or a carbon nanotube. In some embodiments, an antibody or multiple antibodies can be used to detect and capture an amplicon labeled with an optically detectable probe. In some embodiments, an antibody selected to detect a moiety comprising part of a probe with affinity for a specific polynucleotide amplicon may itself be labeled with an optically detectable particle such that binding of the probe by the labeled antibody provides a means of detecting polynucleotide amplicons. For further examples of suitable detection reagents and detection schemes, see PCT/US2012/063190, which is incorporated by reference herein.

Furthermore, the detection zone may incorporate additional features to enhance the ease of detection of an assay result by a user. For example the device may incorporate a control region capable of changing color upon wetting. This color change can be useful to indicate device activation and to serve as a background color to add contrast to a given colorimetric reaction if incorporated directly into the detection zone. Devices of the present invention may also incorporate detection reagents that change into a predictable range of different colors with changing concentration of detected analyte as opposed to varying shades of the same color with changing concentration (e.g., a reagent that changes from red to orange to yellow to green as the concentration of detected analyte increases). This feature can greatly aid in the ability of a user to interpret colorimetric data. In some embodiments of the invention, a timer may be incorporated into the device which serves to indicate to an operator when the device should be read or when the sliding member should be moved to the next station. Such timers have been described by Phillips et al. in Anal. Chem, 2010, 82, 8071-8078, which is incorporated herein by reference in its entirety. Some embodiments of the device may incorporate multiple output zones, each zone being spotted with the same reaction chemistry but in progressively higher concentrations. The concentrations may be chosen such that increasingly higher levels of analyte may be needed to induce a color change in each zone. Thus, the number of zones “activated” will correlate to the amount of analyte in a given sample, resulting in a quantitative readout. In some embodiments of the invention, activation of the detection zone chemistry may result in the appearance of a “plus” sign “+” or “minus” sign “−”. This is accomplished by having a horizontal control line crossed by a vertical sample line. Lines can be generated by printing capture antibodies using plotters, inkjet printers, etc. In this way, a sample which is negative for a particular analyte will only activate the control line and develop a minus “−” symbol while a sample which contains a particular analyte will develop both the horizontal and vertical lines and reveal a plus “+” symbol. The examples above are illustrative of features which may be incorporated into devices of the invention but are in no way exhaustive of the possible features that may be incorporated into such devices.

In one embodiment of the invention, the colorimetric output of the device may be read and interpreted using a cellular phone. Using color intensity analysis software to interpret results enables one to achieve extremely high resolution—even approaching that of an automated method. In addition, interpretation of colorimetric data by this method provides other advantages such as automating inclusion of results in an electronic medical record and facilitating easy transmission for medical decision-making A telemedicine application would also obviate any concerns about color-blind users. A further embodiment of the current invention is the use of cellular phones and accompanying software to meet the following requirements: (i) the system must work on a basic camera phone (such as those common to the developing world); (ii) data gathered by the camera must not be sensitive to camera angle, lighting, or distance from the lens (in preferred embodiments, the paper device contains a color chart which the phone software is able to use for automated calibration); and (iii) the system should be able to automatically recognize the pattern of test zones on the device to minimize user burden. In further embodiments, the device used to record the image is not a cell phone but any device capable of reflectance-based measurement and transmission.

### Assay Reagents

Devices of the present invention may be configured to process biological samples any number of ways depending upon the assay reagents incorporated within or provided to the device during its operation. Assay reagents disposed in the device must be stable in a dry form and able to be activated upon exposure to liquid. Broadly, classes of assay reagents that may be utilized in the operation of devices of the present invention include lysis reagents, wash buffers, nucleotide amplification reagents, reagents that produce exothermic reactions, analyte capture reagents, and detection reagents. The majority of these reagents are discussed in relation to the larger device components with which they are associated.

In some embodiments, stabilizers may be added to the reagent zones to further stabilize the enzymes spotted onto the paper. In further embodiments the stabilizers include but are not limited to: trehalose, poly(ethylene glycol), poly(vinyl alcohol), poly(vinyl pyrrolidone), gelatin, dextran, mannose, sucrose, glucose, albumin, poly(ethylene imine), silk, and arabinogalactan. In some embodiments, dye stabilizers such as MgCl₂ or ZnCl₂ may be added to the assays.

In preferred embodiments, the stabilizers are sugars. A particularly useful method for stabilizing enzymes and other proteins, vacuum foam drying, is described by Bronshtein et al. in U.S. Pat. No. 6,509,146, which is incorporated herein by reference in its entirety.

Desirably an assay reagent is used that provides a colorimetric indication of the presence or amount of an analyte in a sample. When the assay reagent is a fluorescent agent (e.g., SYBR Green I), ultra-violet light may be applied to the fluorescent agent to achieve a colorimetric indication of the presence or amount of an analyte in a sample. Alternatively, the assay reagent may be an electrochemical indicator.

A particularly useful chemistry for measurement of AST and ALT in a blood sample are known AST and ALT assays. The AST assay chemistry utilizes AST present in a sample to convert cysteine sulfinic acid and alpha-ketoglutaric acid to L-glutamic acid and beta-sulfinyl pyruvate. The beta-sulfinyl pyruvate reacts with water to yield free SO₃⁻ which further reacts with methyl green, a blue-colored dye, to yield a colorless compound. This reaction is performed against a pink contrast dye, created by also spotting Rhodamine B onto the paper. As the reaction proceeds, and the dye becomes converted to a transparent compound, more of the pink background is revealed. The visual result is that the detection zone changes from a dark blue to a bright pink color in the presence of AST.

The ALT assay chemistry is based on the conversion by ALT of L-alanine and alpha-ketoglutaric acid to pyruvate and L-glutamic acid, the subsequent oxidation of pyruvate by pyruvate oxidase to form acetyl phosphate and hydrogen peroxide, and the utilization of the liberated hydrogen peroxide by horseradish peroxidase to generate a red-colored dye 4-N-(1-imino-3-carboxy-5-N,N dimethylamino-1,2-cyclohexanediene) through the coupling of 4-amino antipyrine and N,N-dimethylaminobenzoic acid. In further embodiments, the pyruvate generated in the AST chemistry could be used in the same reaction cascade as in the ALT assay as described in U.S. Pat. No. 5,508,173.

Huang et al. describe several methods for transaminase detection in Sensors 2006; 6(7):756-782, which is hereby incorporated by reference in entirety. Additionally, Anon et al. describe methods for AST and ALT detection in Scand. J. Clin. Lab. Invest. 1974; 33(4):291-306, which is hereby incorporated by reference in entirety.

Additional agents that may be incorporated into one or more layers of the device, or alternatively, added to the device via an inlet include, for example, a blocking agent, enzyme substrate, specific binding reagent such as an antibody or sFv reagent, labeled binding agent, e.g., labeled antibody. Such agents may be disposed in the device within or in flow communication with a hydrophilic region. The binding agent, e.g., antibody, may be labeled with an enzyme or a colored particle to permit colorimetric assessment of analyte presence or concentration. Where an enzyme is involved as a label, e.g., alkaline phosphatase (ALP) or horseradish peroxidase (HRP), an enzyme substrate may be disposed in the device within or in flow communication with a hydrophilic region. Exemplary substrates for ALP include 5-bromo-4-chloro-3-indolyl phosphate and nitro blue tetrazolium (BCIP/NBT), and exemplary substrates for HRP include 3,3′,5,5′-Tetramethylbenzidine (TMB), 3,3′-diaminobenzidine (DAB), and 2,2′-azino-bis(3-ethylbenzthiazoline-6-sulphonic acid) (ABTS).

### Timer

A timer may be incorporated into the device which serves to indicate to an operator when the device should be read. Such timers have been described by Phillips et al. in Anal. Chem, 2010, 82, 8071-8078 which is incorporated herein by reference.

In further embodiments, a timer takes the form of a multi-layer device containing a channel of defined length and width such that fluid takes a predictable amount of time to travel to the end of the channel. Upon addition of sample to the device, fluid immediately begins to wick down the defined paper channels. As the fluid wets the channel, it can reveal printed messages on the reverse side of the paper as the paper becomes wet, and therefore transparent.

In some embodiments, a positive control can act as a timer for the test in that when the positive control is fully developed, the device can be read. In further embodiments, the assay may be sensitive to heat or humidity leading to an acceleration or deceleration of the assay. In this situation, a positive control can be tailored such that it exhibits the same acceleration or deceleration effect. In this way, the device is still read when the positive control is developed, and no timer is needed.

Lateral Fluid Flow within a Layer

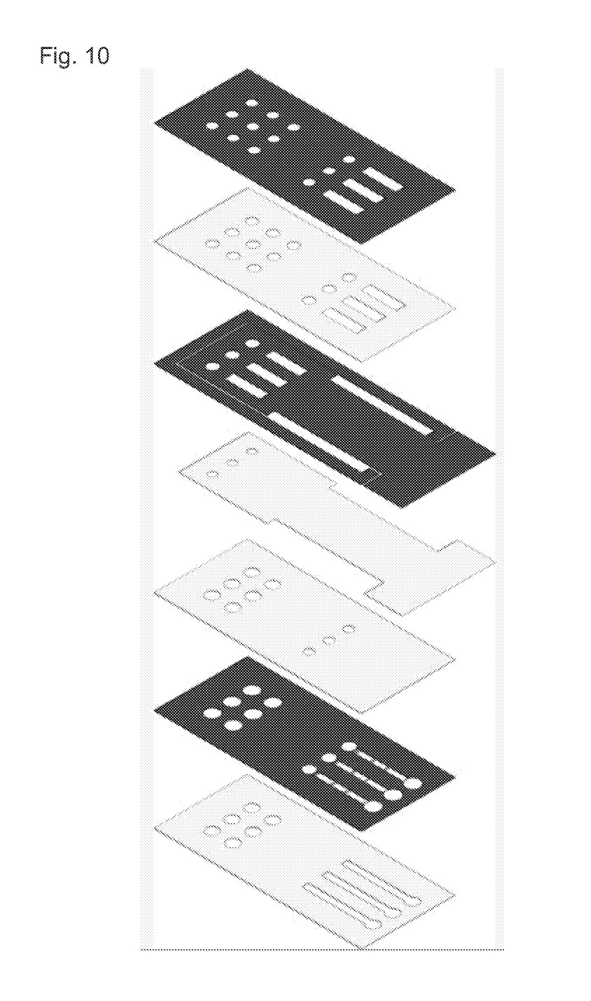

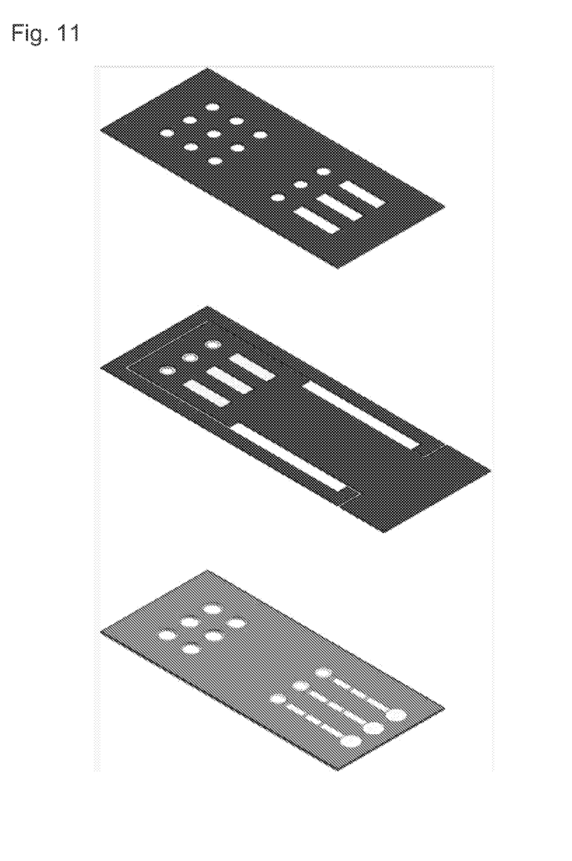

The devices may optionally comprise a region for lateral flow of fluid within a layer of the device. The region for lateral flow of fluid desirably comprises a hydrophilic material, such as paper, cloth, or a polymer film. Flow of fluid within such hydrophilic material may be controlled by hydrophobic barriers penetrating through the hydrophilic material. The region for lateral flow of fluid may be within a magnetic layer, such as the second magnetic layer. FIG. 10 is an exploded view of a microfluidic device showing three magnetic layers and multiple laminate layers, in which the bottom (i.e., third) magnetic layer contains a region for lateral flow of fluid. Rectangular openings in the first magnetic layer and second magnetic layer are ports to permit visualization of lateral fluid flow (and results therefrom) in the third magnetic layer. FIG. 11 is a condensed view of the microfluidic device from FIG. 10 in which laminate layers (e.g., substantially planar, hydrophilic substrates) are bonded to the appropriate magnetic layer.

### Fluid-Impermeable Layer

Some embodiments of the device may comprise one or more fluid-impermeable layers. The fluid-impermeable layers, as opposed to the substantially planar layers that are modified to contain hydrophilic regions defined by fluid-impermeable boundaries, are comprised entirely of hydrophobic materials except for the region through which fluid flows which may be a hole or hydrophilic material through which fluid can pass. Fluid-impermeable layers are typically planar sheets that are not soluble in the fluid of the microfluidic device and provide a desired level of device stability and flexibility. In certain embodiments, the fluid-impermeable layers are plastic sheets, adhesive sheets, or tape. In some embodiments, double-sided tape is used as a fluid-impermeable layer. Double-sided tape adheres to two adjacent layers of porous hydrophilic material (e.g., porous hydrophilic material treated using methods to produce fluid impervious barriers) and may be used to bind to other components of the microfluidic device. The fluid-impermeable barriers are impermeable to water, and isolate fluid streams separated by less than, for example 200 μm. In addition, the fluid-impermeable barriers are sufficiently thin to allow adjacent porous, hydrophilic layers to contact through holes punched in the fluid-impermeable barriers (e.g., perforations) when compressed.

Non-limiting examples of a fluid-impermeable layer include Scotch® double-sided carpet tape, 3M Double Sided Tape, Tapeworks double sided tape, CR Laurence black double sided tape, 3M Scotch Foam Mounting double-sided tape, 3M Scotch double-sided tape (clear), QuickSeam splice tape, double sided seam tape, 3M exterior weather-resistant double-sided tape, CR Laurence CRL clear double-sided PVC tape, Pure Style Girlfriends Stay-Put Double Sided Fashion Tape, Duck Duck Double-sided Duct Tape, and Electriduct Double-Sided Tape. As an alternative to double-sided tape, a heat-activated adhesive can be used to seal the fluid-carrying layers together. Indeed, any fluid-impermeable material that can be shaped and adhered to the pattern hydrophilic layers can be used. Pressure sensitive adhesives may also be deposited onto the paper sheets in a desired pattern. This can be accomplished by printing or stamping the adhesive onto the paper. A particularly useful embodiment involves screen printing of the pressure-sensitive adhesive. In addition, it is possible to use the same material that is used to pattern the paper layers to join the layers of paper together.

### Additional Features

The device may optionally be further characterized by defining in one or more hydrophilic regions a reservoir for receiving a clinical sample; a distributing region for receiving the sample from the reservoir and distributing separate portions of the sample; and plural spaced apart storage regions for receiving the sample from the distributing region.

In some embodiments of the invention, the device may consist of a magnetic layer made of magnetic media and arranged to provide an attractive force suitable to retain or manipulate magnetic assay reagents. Contemplated magnetic assay reagents include magnetized molecular reagents similar to Dynabeads®. Other examples of suitable magnetic reagents include nucleotide compositions, oligonucleotides, small molecules, dyes, antibodies, antibody fragments, or nanoparticles that are themselves magnetized or are linked to suitable magnetized microscopic particles. Such reagents are contemplated to be especially useful in the process of filtering and retaining targeted components of a biological sample.

In addition, the device may be configured for fractionating a small volume (30 μL) of blood to multiple detection zone, wherein each detection zone contains different reagents for performing different diagnostic tests.

Further embodiments of the invention and suitable components and methods of use of the invention described herein are described in Appendices A and B of this document.

### Methods of Using the Devices

Devices described herein may be used to process biological samples and detect the presence or concentration of specific analytes. Accordingly, one aspect of the invention provides a method of providing a device described herein, adding a sample to the device, and detecting the presence of an analyte in the sample.

In some embodiments of the device, a sliding strip layer must be moved serially between different stations of the device and different wash and reaction reagents applied at each station to facilitate processing of the biological sample. Furthermore, some embodiments of the invention require the user to apply heat or power in order to facilitate different reaction steps. Operation of the device may also require the user to wait specified periods of time between different reaction steps in order to allow individual reactions to reach completion. In some embodiments of the device, the user will need to analyze a test zone or detection zone of the device in order to determine the presence, quantity, or concentration of an analyte in a processed sample. Depending upon the specific detection reagent or reagents incorporated in a particular embodiment of a device of the invention, detection may be accomplished visually (i.e., by eye) or with the aid of external devices capable of reading an optical signal (e.g., a mobile telephone camera or fluorometer). Furthermore, in some embodiments of the device, detection will require a user to remove the device housing, outer magnetic strip members, other substantially planar members, or a sliding planar member in order to expose internal components of the device containing the detection reagent or test zone.

### DEFINITIONS

To facilitate an understanding of the present invention, a number of terms and phrases are defined below.

The terms “a,” “an” and “the” as used herein mean “one or more” and include the plural unless the context is inappropriate

Throughout the description, where devices are described as having, including, or comprising specific components, or where processes and methods are described as having, including, or comprising specific steps, it is contemplated that, additionally, there are devices of the present invention that consist essentially of, or consist of, the recited components, and that there are processes and methods according to the present invention that consist essentially of, or consist of, the recited processing steps.

### EXAMPLES

The invention now being generally described, will be more readily understood by reference to the following examples, which are included merely for purposes of illustration of certain aspects and embodiments of the present invention, and are not intended to limit the invention.

### Example 1

### Preparation of Microfluidic Device Containing Magnetic Media

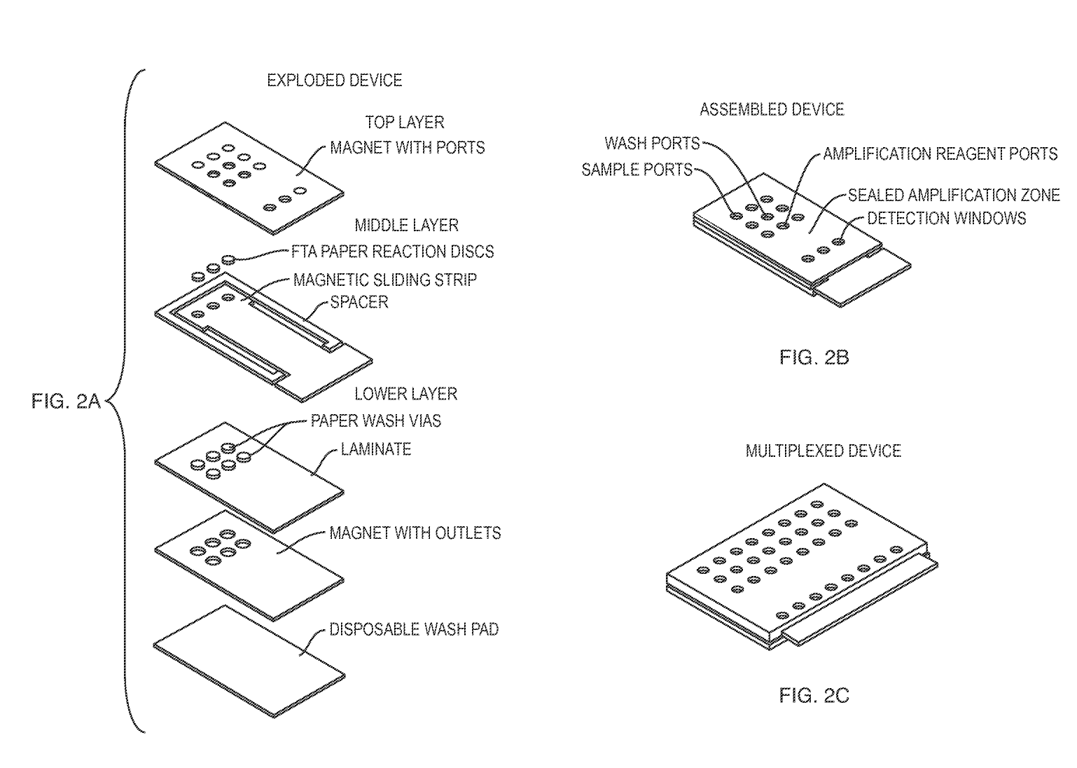

A microfluidic device containing magnetic media was prepared according to the procedures described below. The microfluidic device contained three layers of magnetic media. The second magnetic layer contained a sliding strip. An exploded view of the device is provided in FIG. 2a. The assembled device is depicted in FIG. 2b. The device contains three major layers: the top layer, which provides ports to access reaction discs; the middle layer, which consists primarily of the reaction discs housed in the sliding strip that is linearly actuated to serially address the ports; and the bottom layer, which contains paper vias to contact the reaction disc and the wash pad to transit waste wash out of the device. Each of these layers is fabricated out of a low-cost, flexible magnetic substrate, the attractive force of which, when combined with a thin film of inert lubricant, creates a dynamic seal that allows for extended incubation at 65° C. with negligible evaporation.

Because the device contains three reactions discs, a first reaction disc may be used to process the sample, while the second and third reaction discs may be used as a negative control (NC) and a positive control (PC), respectively. As illustrated in FIG. 2b, the top layer of magnetic media desirably contains ports to add the samples, wash buffer, amplification Master Mix (MM), and detection reagents and to visualize the signal (Read). The magnetic layers used to fabricate the device, in conjunction with an inert lubricant, minimizes evaporation of fluid (e.g., water) from the device.

To allow for multiple samples—replicates, controls, or different samples—to be run in parallel, the architecture can be scaled to a multiplexed format as depicted in FIG. 2C.

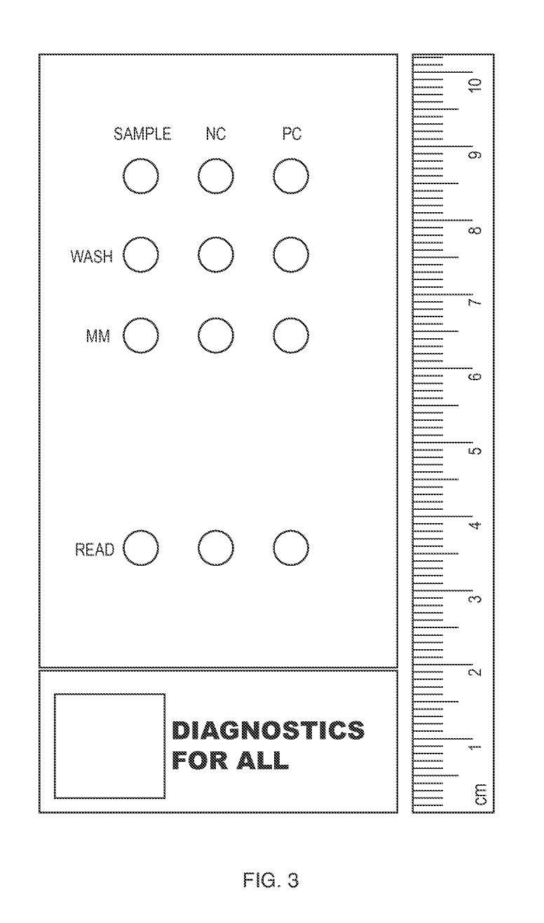

The devices are generally small, such as only 0.225 cm thick. One exemplary device, depicted in FIG. 3, is capable of running one sample and two controls. This device is 4.7 cm wide and 10 cm from the top to the end of the strip-pull when it is in the starting position. The device may be used with an inexpensive handheld instrument that will automate the linear motion and power heating. Lyophilized reagents may be stored within the device.

### Procedures:

Magnetic material used to fabricate the device was 0.25 mm thick synthetic rubber-bonded ferrite magnetic sheeting with 3 mm bands of alternating poles on one face and an adhesive backing on the other obtained from McMaster-Carr (Robbinsville, N.J.). Three layers of this material were laminated together to assure correct alignment of poles and to achieve the desired thickness. The magnetic sheets, as well as all laminate layers, were cut using a design file from Adobe Illustrator and a Graphtec CraftRoboPro (Graphtec Irvine, Calif.) knife plotter. The reaction disc was Whatman FTA® paper having a diameter 4.75 mm. Wash vias were discs having a diameter of 6.35 mm that were cut from Ahlstromm 226 paper. The various layers were stacked, laminated, and pressed as illustrated in FIG. 2a.

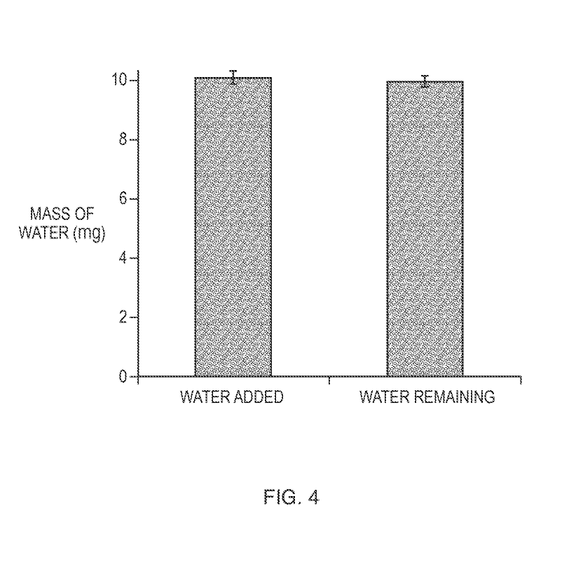

The attractive force produced by the magnetic material forms an evaporation-resistant seal in combination with a thin film of Krytox® LVP high performance lubricant (Dupont). This seal was tested by applying 10 μL of water to the paper reaction disc within a sliding strip device, which was pre-dried to remove any ambient moisture, and slid to seal. The mass of the device before and after the addition of the water was recorded to serve as the baseline. The device was then incubated at 65° C. for 1 hour, weighed, and slid to unseal the disc. The device was returned to the incubator to completely evaporate any water remaining and the final mass recorded. The masses of the device after incubation and after complete evaporation were used to determine the amount of water lost during incubation by comparison to the baseline. Experimental results in FIG. 4 show that an average loss of less than 0.02 μL of 10 μL water applied (n=4) after 1 hour of heating the device at 65° C. occurred.

### CONCLUSION

A microfluidic device containing magnetic media was prepared to enable sample preparation from clinically-relevant matrices, enable isothermal amplification of analyte in the same, and detect the analyte. The cost of materials in the device was approximately $0.59 USD. As illustrated in FIG. 2a, the multi-layer architecture of device, allows for the middle layer—the strip—containing the reaction vessel, a paper reaction disc, to slide into different fluidic paths. This linear motion acts as a valve to control the serial introduction of sample, wash buffer, amplification reagents, and detection reagents, while also dynamically sealing to prevent evaporation during reaction incubation. Further, the sample preparation steps are simplified by fabricating the reaction disc out of Whatman FTA® paper, which contains a proprietary blend of lytic reagents dried into the cellulose matrix. FTA® paper has been shown to lyse a wide range of cells and viruses.

The microfluidic device of the invention containing magnetic media replaces instrumented, multistage, high-volume washing required by procedures in the literature for LAMP reactions with two, through-flow washes before the introduction of LAMP master mix. Also, by exploiting the bibulous nature of paper, we simplify fluid handling by replacing complex pumping systems from literature procedures with fluid flow via capillary action. Final detection of the amplified product can be achieved using SYBR Green I, a fluorescent intercalating dye, and a handheld UV source and camera phone.

### Example 2

### Analytical Use of Microfluidic Device Containing Magnetic Media

Analytical performance of the microfluidic device from Example 1 was evaluated in the tests described in Part I below. Experimental procedures are described in Part II below.

### Part I: Analytical Performance Tests

### Analytical Sensitivity in Paper

To assess the performance of the LAMP reaction when conducted within a paper disc that is housed in the sliding strip of the device in Example 1, serial dilutions of the 200 bp dsDNA target sequence in nuclease-free water were analyzed. By drying these samples onto reaction discs which had been treated with human plasma and washed, we replicated the condition of the discs that would exist in the full assay prior to amplification and isolated potential amplification inhibition from sample preparation loses.

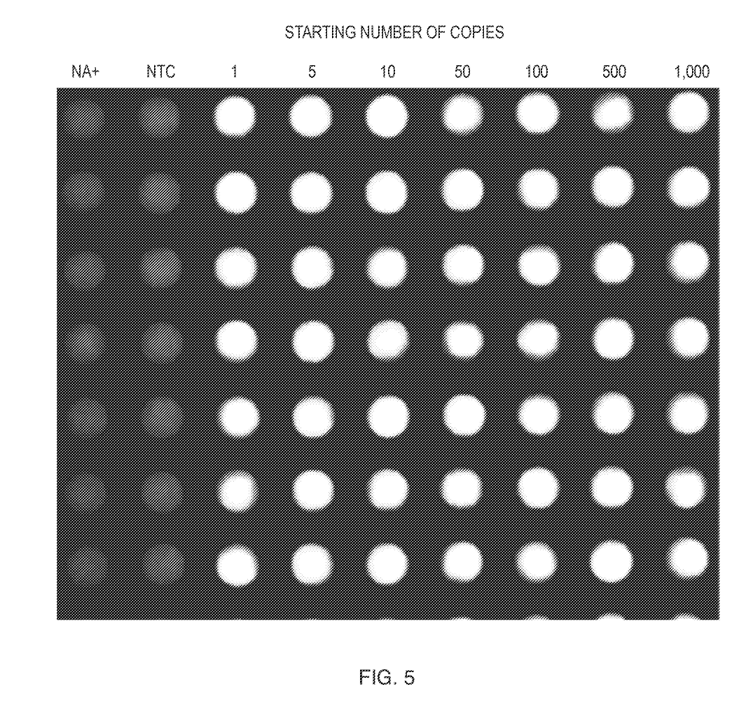

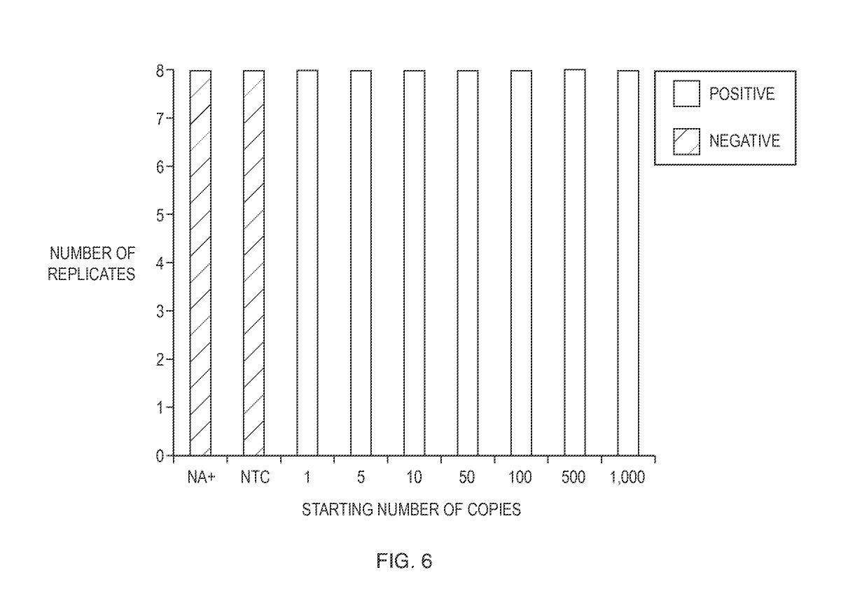

Performance of the LAMP reaction can be visualized by applying SYBR Green I (e.g., 10 μL of 100×SYBR Green I) to the reaction disc after the LAMP reaction is complete, then exposing the disc to ultra-violet radiation. The resulting fluorescence can be photographed using a camera phone. A camera phone image of the results from this experiment are depicted in FIG. 5. A bar graph showing analytical sensitivity of LAMP in the device is depicted in FIG. 6, where positive replicates are those producing signals greater than the limit of detection (LOD) calculated as the average plus three times the standard deviation of the no template control (NTC) (n=8).

Though no sequence homology exists between primers in the set in this experimental, it is contemplated that mismatches would be tolerated. To confirm that the fluorescent signal was the result of the specific template-driven reaction, a restriction endonuclease analysis was performed.

Integrated Assay with Spiked Plasma Samples

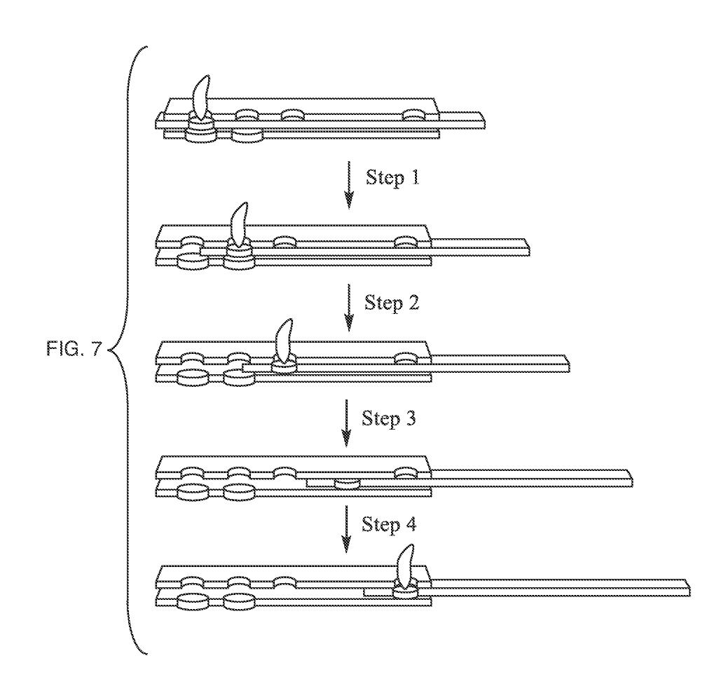

The assay above was challenged with whole, live E. coli serially diluted in human plasma. The samples were subjected to all of the steps of sample preparation by linear actuation of the sliding strip to the appropriate ports for application of the wash buffer, LAMP Master Mix, and detection reagents, as illustrated in FIG. 7 where: Step 1 is to apply sample to the reaction disc; Step 2 is to slide the strip to move the reaction disc to a wash port and apply wash buffer to reaction disc; Step 3 is to slide the strip to move the reaction disc to a reagent port and apply reagent mix (e.g., Master Mix) to the reaction disc; Step 4 is to slide the strip to a sealed amplification zone and incubate the device (e.g., heat the device to elevated temperature); and Step 5 is to slide the strip to move the reaction disc to a detection window and apply a detection reagent (e.g., SYBR Green I followed by exposure to ultra-violet radiation) and visualize the results (e.g., by taking a picture using a camera phone). As before, the replicates were binned as positive or negative, based on mean gray values quantified via ImageJ (n=6).

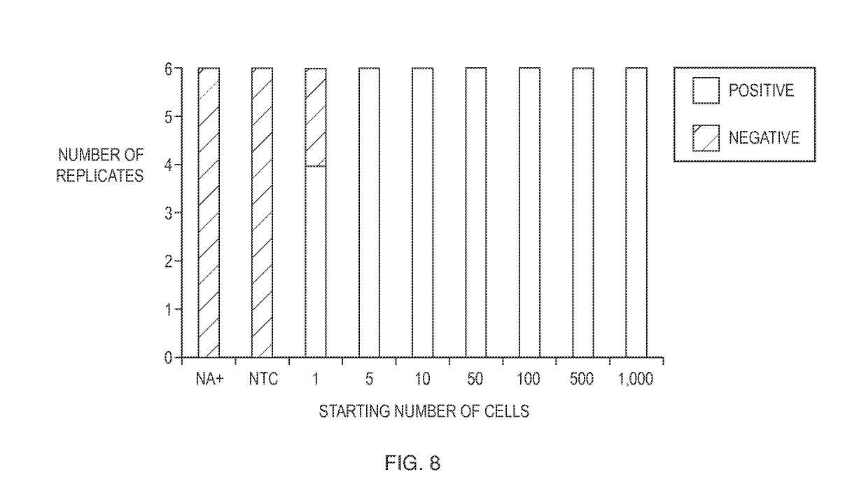

Positive results were obtained from four of six samples containing as few as 1 cell (FIG. 8). This outcome is consistent with stochastic sample error as predicted by the most probable number (MPN) method. Statistical significance (p<0.001) was achieved for all concentrations over 5 cells per sample. These results are shown to be the result of template-specific amplification via agarose gel electrophoresis (AGE) as they all exhibit the expected banding pattern of the correct LAMP product.

Collecting and Concentrating DNA from a Larger Sample Volume

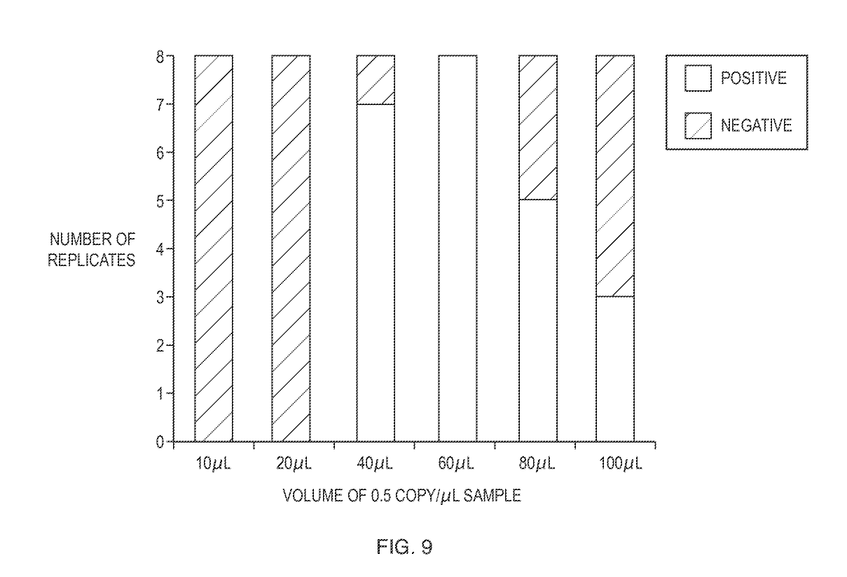

In instances when the analyte of interest is present at very low concentrations, it can be beneficial to sample a larger volume in order to increase the likelihood of capturing more copies, thus concentrating the target in the reaction disc. Accordingly, the effects of a larger sample volume were investigated. By applying a sample (that contained 0.05 E. coli cells/μL of fluid) in increments of 10 μL, we observed an increase in the number of replicates resulting in positive amplification up to a certain threshold. Specifically, 10 μL and 20 μL samples (approximately 0.5 and 1 cell applied to the discs, respectively) produced no positive signals, a 40 μL sample (approximately 2 cells per disc) produced 7 positive replicates, and a 60 μL sample (approximately 3 cells applied to the discs) yielded positive signals in all 8 replicates. The results are displayed graphically in FIG. 9. This demonstrates the ability to concentrate DNA in the reaction disc.

However, further increases in volume did not increase performance. In fact, a fluid volume over 60 μL degraded the gains noted above, with 80 μL and 100 μL samples (approximately 4 and 5 cells applied to each disc, respectively) dropping to 5 and 3 positive replicates, respectively. This decline may be due to washing effects, both washing away of the dried lysis chemistry of the FTA® paper disc, preventing the release of additional target, and washing away of previously captured target.

### Part II: Experimental Procedures

### Materials

Oligonucleotide primers were ordered from Eurofins Operon MWG (Huntsville, Ala.) with HPLC purification and suspended in 1×TE. Bst 2.0 DNA polymerase, 10×Isothermal Amplification Buffer, dNTPs, DNA/DNAse—free stocks of MgSO₄, and restriction endonucleases were purchased from New England Biolabs (Ipswich, Mass.) and betaine from Sigma-Aldrich (St. Louis, Mo.). Pooled human plasma in sodium heparin derived from whole blood donations was acquired from Valley Biomedical Products and Services, Inc. (Winchester, Va.). Pre-cast 2% agarose gels and SYBR Green I were purchased from Invitrogen. All other reagents were supplied by Fisher Scientific.

E. coli Culture

E. coli BL21(DE3)pLysS was used as a model organism. Cells were cultured overnight at 37° C., shaking, from frozen glycerol stocks in LB broth containing 34 μg/mL chloramphenicol. The concentration of cells was determined by measuring the optical density at 600 nm and comparing this to a growth curve. When directly used to spike sample, the culture was centrifuged, the supernatant aspirated, and the pellet re-suspended to the desired concentration with fresh media, twice, to remove any free DNA. This stock of cells was then serially diluted in human plasma for experiments.

### LAMP Amplification

The primer set targeting the malB gene of E. coli, including loop primers, as reported by Hill et al. were employed. Hill et al. (2008) J. Clin. Microbiol. 46(8):2800-2804. LAMP was performed using the Bst 2.0 DNA Polymerase, an in silico designed homologue of Bst DNA Polymerase I, Large Fragment, commonly used in this reaction, and its supplied buffer. In transitioning the reaction from tubes to paper, the concentration of betaine, primers, and enzyme were optimized, as was the incubation time to minimize non-specific reactions and maximize sensitivity. The final reactions contained 1× Isothermal Amplification Buffer (10 mM Tris-HCl, 10 mM (NH₄)₂SO₄, 50 mM KCl, 2 mM MgSO₄, and 0.1% Tween-20) supplemented with an additional 6 mM MgSO₄, 0.9 M betaine, 1.4 mM each dNTP, 1.6 μM each inner primer (FIP, BIP), 0.8 μM each loop primer (LF, LB), 0.2 μM each outer primer (F3, B3), and 0.32 U of polymerase. The optimal incubation was 65° C. for 60 min. The reaction was performed with two negative controls—a no template control (NTC) and a no amplification control (NA+), containing a heat inactivated enzyme and 1,000 copies of target or 1,000 cells, as appropriate. Reactions in tubes were carried out at 25 μL, whereas in paper the reaction volume was 10 μL.

### Product Analysis by Gel Electrophoresis

To confirm that positive signals were the result of specific, template-driven reactions, LAMP products were analyzed by agarose gel electrophoresis (AGE). First, a positive reaction conducted in a tube using 1,000 copies of the target was digested using restriction endonuclease PvuII, which recognizes a sequence in region B1 of the target, and run on a 2% agarose gel with a DNA ladder and the un-digested product to determine if the observed LAMP banding pattern was the result of template-specific amplification and to mark said banding pattern as the specific reaction product for future comparison. Second, using this result, AGE analysis was performed directly from reaction discs by folding them in half and inserting them into the wells of the gel and observing the resulting band pattern.

### General Sliding Strip Operation

The sliding strip devices enable the serial operations of sample preparation—including cell lysis, DNA isolation and purification—as well as LAMP amplification and detection. Unless otherwise noted, the procedure for sliding strip assays, illustrated in FIG. 7, began with the application to the reaction disc of 10 μL sample through the sample port. The strip was then slid to align the reaction disc with the wash port and 40 μL of FTA Purification Buffer and 80 μL of nuclease-free water were sequentially applied and allowed to transit through the disc to the paper vias and finally to the removable wash pad. The disc was then dried completely, at 65° C. for 10 min, the strip slid to align the disc with the amplification reagent port, and 10 μL of LAMP Master Mix was applied. The strip was then slid again, sealing the disc within the amplification zone by the magnetic force and the lubricant film, and placed in an incubator at 65° C. After a 60 min incubation time, the devices were removed from the incubator and the strip was slid to the detection window, and the disc was dried completely.

For detection, 10 μL of 100×SYBR Green I in 1×TE was applied to the disc and it was excited using a handheld shortwave UV source and imaged using a camera phone. Images were then processed, converted to grayscale, and analyzed using Image J to quantify mean pixel intensity as gray value. The mean of the NTC replicates plus 3 times their standard deviation was used as the LOD, and the threshold value for determining positive and negative reactions. In some cases, for ease of imaging and data presentation of multiple samples and replicates, the reaction discs were removed from the device and arrayed before imaging.

### Determination of Analytical Sensitivity

Analytical sensitivity of amplification and detection in the sliding strip device was determined using a double-stranded 200 bp analog of the malB target sequence produced by Integrated DNA Technologies, Inc (Coralville, Iowa) as two single-stranded DNA Ultramers®, the forward strand and the reverse complement. These were then combined to yield an equimolar solution, heated to 95° C. for 5 min, and then slowly cooled to room temperature to anneal. The concentration was confirmed by spectrometry using the absorbance at 280 nm. Serial dilutions were prepared and 10 μL applied to dry reaction discs, which had been pretreated with 10 μL human plasma, 40 μL FTA Purification Buffer, and 80 μL nuclease-free water, through the amplification reagent port of the sliding strip devices. The discs were then dried completely, and the general sliding strip operations outlined above were followed starting from the application of LAMP Master Mix.

Integrated Assay with Spiked Plasma Samples

To examine effectiveness of the sliding strip device for sample preparation from complex matrices, the assay was challenged with samples of human plasma spiked with whole, live E. coli prepared from overnight culture as described above. All steps in the general sliding strip operation outlined previously were followed. Parameters such as the number of washes, wash volumes, and wash buffer composition were optimized.

Collecting and Concentrating DNA from a Large Sample Volume

To examine the ability to isolate DNA from larger volumes of dilute samples, we applied 10, 20, 30, 40 or 50 μL of a 0.05 cell/μL sample of E. coli in human plasma to the reaction discs. After each sample wicked through the reaction disc, the general sliding strip operation procedure was followed as described.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A three-dimensional microfluidic device comprising:
a first magnetic layer comprising magnetic media, a first inlet through which fluid can pass, and a reagent inlet through which fluid can pass; and
a second magnetic layer comprising (i) magnetic media configured to provide an attractive force between said first magnetic layer and said second magnetic layer, and (ii) a hydrophilic region;
wherein the second magnetic layer is movable relative to the first magnetic layer to permit establishment of fluid flow communication serially between an inlet in the first magnetic layer and the hydrophilic region in the second magnetic layer.

2. The device of claim 1, further comprising a first substantially planar member comprising a hydrophilic region in fluid communication with the hydrophilic region in the second magnetic layer, wherein said first substantially planar member is located adjacent to the second magnetic layer opposite the first magnetic layer.

3. The device of claim 2, wherein the first substantially planar member is a hydrophilic material comprising a fluid-impermeable barrier that defines a boundary of a hydrophilic region.

4. The device of claim 2 or 3, wherein the first substantially planar member comprises a material selected from the group consisting of paper, cloth, and polymer film.

5. The device of claim 2 or 3, wherein the first substantially planar member comprises paper.

6. The device of claim 1, further comprising a third magnetic layer comprising (i) magnetic media configured to provide an attractive force between said second magnetic layer and said third magnetic layer, and (ii) an outlet through which fluid can pass, the third magnetic layer being located adjacent to the second magnetic layer opposite the first magnetic layer.

7. The device of any one of claims 2-5, further comprising a third magnetic layer comprising (i) magnetic media configured to provide an attractive force between said second magnetic layer and said third magnetic layer, and (ii) an outlet through which fluid can pass, the third magnetic layer being located adjacent to the first substantially planar member opposite the second magnetic layer.

8. The device of claim 6 or 7, further comprising a second substantially planar member comprising a hydrophilic region in fluid communication with an outlet in the third magnetic layer, wherein said second substantially planar member is located adjacent to the third magnetic layer opposite the second magnetic layer.

9. The device of claim 8, wherein the second substantially planar member comprises a material selected from the group consisting of paper, cloth, and polymer film.

10. The device of claim 8, wherein the second substantially planar member comprises paper.

11. The device of any one of claims 1-10, further comprising a track housing the second magnetic layer, the track comprising substantially planar members along which the second magnetic layer may slide laterally.

12. The device of claim 11, wherein the track comprises magnetic media configured to provide an attractive force between the first magnetic layer and the track.

13. The device of claim 12, wherein the track and second magnetic layer both comprise a double-sided magnetic sheet.

14. The device of any one of claims 1-13, wherein the first inlet is for receiving a sample.

15. The device of any one of claims 1-14, wherein the first magnetic layer further comprises an inlet for receiving a wash buffer, wherein establishment of fluid communication between said inlet for receiving a wash buffer and said hydrophilic region in said second magnetic layer is effected by movement of said second magnetic layer relative to the first magnetic layer.

16. The device of any one of claims 1-15, wherein the first magnetic layer further comprises an inlet for receiving an analyte amplification reagent, wherein establishment of fluid communication between said inlet for receiving an analyte amplification reagent and said hydrophilic region in said second magnetic layer is effected by movement of said second magnetic layer relative to the first magnetic layer.

17. The device of any one of claims 1-16, wherein the reagent inlet is for receiving an analyte detection reagent, wherein establishment of fluid communication between said inlet for receiving an analyte detection reagent and said hydrophilic region in said second magnetic layer is effected by movement of said second magnetic layer relative to the first magnetic layer.

18. The device of any one of claims 1-17, further comprising a site bounded by a seal for inhibiting evaporation of fluid from the device.

19. The device of claim 18, wherein the seal comprises a grease.

20. The device of claim 18 or 19, wherein less than 1% w/w of water contained in the site bounded by a seal evaporates when the device is heated to 65° C. for a duration of 1 hour.

21. The device of any one of claims 1-20, wherein the first magnetic layer contains an inlet through which fluid can pass that leads to a positive control zone in the second magnetic layer.

22. The device of any one of claims 1-21, wherein the first magnetic layer contains an inlet through which fluid can pass that leads to a negative control zone in the second magnetic layer.

23. The device of any one of claims 1-22, wherein the hydrophilic region in the second magnetic layer comprises paper.

24. The device of any one of claims 1-23, wherein the hydrophilic region in the second magnetic layer comprises a reagent that causes a cell to lyse.

25. The device of any one of claims 1-24, wherein the hydrophilic region in the second magnetic layer comprises one or more of uric acid or a salt thereof, a detergent, a base, and a chelating agent.

26. The device of any one of claims 1-25, wherein a filter is present in the sample inlet in the first magnetic layer.

27. The device of any one of claims 1-26, wherein the magnetic media comprises ferrite.

28. The device of any one of claims 1-27, wherein the magnetic media comprises a mixture of ferrite and a binder selected from the group consisting of synthetic vinyl rubber, poly(dimethylsiloxane), a polyurethane, a natural rubber, a fluoroelastomer, and combinations thereof.

29. A three-dimensional microfluidic device comprising:
a first magnetic layer comprising magnetic media and a sample inlet through which fluid can pass;
a second magnetic layer comprising magnetic media configured to provide an attractive force between said first magnetic layer and said second magnetic layer; and
a first substantially planar member comprising a fluid-impermeable barrier that defines a boundary of at least one hydrophilic region in fluid communication with said sample inlet, wherein said first substantially planar member is disposed between the first magnetic layer and the second magnetic layer.

30. The device of claim 29, further comprising a second substantially planar member comprising a fluid-impermeable barrier that defines a boundary of at least one hydrophilic region, wherein said second substantially planar member is located adjacent to the first substantially planar member.

31. The device of claim 30, wherein the first substantially planar member and the second substantially planar member are moveable relative to each other to permit establishment of fluid flow communication serially between a hydrophilic region of said first substantially planar member and a hydrophilic region of said second substantially planar member.

32. The device of claim 29 or 31, wherein a hydrophilic region in the second substantially planar member comprises an assay reagent.

33. The device of claim 32, wherein the assay reagent provides a colorimetric indication of the amount of analyte present in a sample.

34. The device of any one of claims 29-33, wherein the second magnetic layer comprises a transparent region for viewing colorimetric indication of the amount of analyte present in a sample.

35. A three-dimensional microfluidic device comprising:
a first magnetic layer comprising magnetic media and a sample inlet through which fluid can pass;
a first substantially planar member adjacent to the first magnetic layer and comprising a fluid-impermeable barrier that defines a boundary of at least one hydrophilic region that is in fluid communication with the sample inlet of the first magnetic layer;
a second magnetic layer comprising a region through which fluid can pass and magnetic media configured to provide an attractive force between said first magnetic layer and said second magnetic layer; said second magnetic layer being located adjacent to the first substantially planar member on the side of the first substantially planar member opposite said first magnetic layer, and said second magnetic layer being moveable relative to the first substantially planar member to permit establishment of fluid flow communication serially between a hydrophilic region of said first substantially planar member and the region of said second magnetic layer through which fluid can pass;
a second substantially planar member disposed on the side of the second magnetic layer opposite said first substantially planar member, said second substantially planar member comprising a fluid-impermeable barrier that defines a boundary of at least one hydrophilic region that can be in fluid communication with the region of the second magnetic layer through which fluid can pass depending on the position of the second magnetic layer; and
a third magnetic layer comprising magnetic media configured to provide an attractive force between said second magnetic layer and said third magnetic layer, wherein said third magnetic layer is disposed on the side of the second substantially planar member opposite the second magnetic layer.

36. The device of claim 35, further comprising a third substantially planar member disposed between the second magnetic layer and the second substantially planar member, wherein the third substantially planar member comprises a fluid-impermeable barrier that defines a boundary of at least one hydrophilic region that is always in fluid communication with a region of the second magnetic layer through which fluid can pass.

37. The device of claim 35 or 36, further comprising a track housing the second magnetic layer, the track comprising substantially planar members along which the second magnetic layer may slide laterally.

38. The device of claim 37, wherein the track comprises magnetic media configured to provide an attractive force between the first magnetic layer and the track, and an attractive force between the third magnetic layer and the track.

39. The device of claim 37 or 38, wherein the track and second magnetic layer both comprise a double-sided magnetic sheet.

40. The device of any one of claims 35-39, wherein the first magnetic layer further comprises an inlet for receiving an analyte amplification reagent, said inlet being in fluid communication with a hydrophilic region specific for an analyte amplification reagent in the first substantially planar member.

41. The device of any one of claims 35-40, wherein the first magnetic layer further comprises an inlet for receiving a wash buffer, said inlet being in fluid communication with a hydrophilic region specific for a wash buffer in the first substantially planar member.

42. The device of any one of claims 35-41, wherein the first magnetic layer further comprises an inlet for receiving an analyte detection reagent, said inlet being in fluid communication with a hydrophilic region specific for an analyte detection reagent in the first substantially planar member.

43. The device of any one of claims 35-42, wherein the region through which fluid can pass in the second magnetic layer comprises a hydrophilic medium.

44. The device of any one of claims 35-43, wherein the region through which fluid can pass in the second magnetic layer comprises paper.

45. The device of any one of claims 35-43, wherein a hydrophilic region in the second substantially planar member comprises an assay reagent.

46. The device of claim 45, wherein the assay reagent provides a colorimetric indication of the amount of analyte present in a sample.

47. The device of any one of claims 35-46, wherein the third magnetic layer comprises a transparent region for viewing colorimetric indication of the amount of analyte present in a sample.

48. The device of any one of claims 35-47, wherein the second substantially planar member comprises a material selected from the group consisting of paper, cloth, and polymer film.

49. The device of any one of claims 35-48, wherein the first substantially planar member comprises a material selected from the group consisting of paper, cloth, and polymer film.

50. The device of any one of claims 35-48, wherein the first substantially planar member comprises paper.

51. The device of any one of claims 35-50, wherein the fluid-impermeable barriers comprise a wax, poly(methylmethacrylate), an acrylate polymer, polystyrene, polyethylene, polyvinylchloride, a fluoropolymer, a photoresist, or a photo-polymerizable polymer that forms a hydrophobic polymer.

52. The device of any one of claims 35-51, wherein the fluid-impermeable barriers that define boundaries of said plural hydrophilic regions are produced by screening, stamping, printing or photolithography.

53. The device of any one of claims 35-52, further comprising a site bounded by a seal for inhibiting evaporation of fluid from the device.

54. The device of claim 53, wherein the seal comprises a grease.

55. The device of any one of claims 35-54, wherein a filter is present in the sample inlet in the first magnetic layer.

56. The device of any one of claims 35-55, wherein the magnetic media comprises ferrite.

57. The device of any one of claims 35-55, wherein the magnetic media comprises a mixture of ferrite and a binder selected from the group consisting of synthetic vinyl rubber, poly(dimethylsiloxane), a polyurethane, a natural rubber, a fluoroelastomer, and combinations thereof.

58. A three-dimensional microfluidic device comprising:
a first substantially planar member comprising a fluid-impermeable barrier that defines a boundary of at least one hydrophilic region; and
a magnetic layer comprising magnetic media, said magnetic layer being located adjacent to and in fluid communication with a hydrophilic region of the first substantially planar member, and the magnetic layer being moveable relative to the first substantially planar member.

59. The device of claim 58, further comprising a second substantially planar member comprising a fluid-impermeable barrier that defines a boundary of at least one hydrophilic region; said second substantially planar member being located adjacent to the magnetic layer on the side opposite the first substantially planar member.

60. The device of claim 58 or 59, wherein the magnetic layer comprises a fluid-impermeable barrier that defines a boundary of at least one hydrophilic region that can be in fluid communication with a hydrophilic region of said first substantially planar member.

61. The device of any one of claims 58-60, wherein the first substantially planar member comprises a material selected from the group consisting of paper, cloth, and polymer film.

62. The device of any one of claims 58-60, wherein the first substantially planar member comprises paper.

63. The device of any one of claims 58-62, wherein the fluid-impermeable barriers comprise a wax, poly(methylmethacrylate), an acrylate polymer, polystyrene, polyethylene, polyvinylchloride, a fluoropolymer, a photoresist, or a photo-polymerizable polymer that forms a hydrophobic polymer.

64. The device of any one of claims 58-63, wherein the fluid-impermeable barriers that define boundaries of said plural hydrophilic regions are produced by screening, stamping, printing or photolithography.

65. The device of any one of claims 58-64, wherein the magnetic media comprises ferrite.

66. The device of any one of claims 58-65, wherein the magnetic media comprises a mixture of ferrite and a binder selected from the group consisting of synthetic vinyl rubber, poly(dimethylsiloxane), a polyurethane, a natural rubber, a fluoroelastomer, and combinations thereof.

67. The device of any one of claims 58-66, wherein a filter is present in the region through which fluid can pass in the first magnetic layer.

68. A three-dimensional, microfluidic assay device for detection of analytes by applying a fluid sample onto at least one substantially planar member comprising a hydrophilic region containing one or more test zones defined by a fluid-impermeable barrier, the improvement comprising a first magnetic layer comprising magnetic media and a sample inlet through which fluid can pass to a hydrophilic region of at least one substantially planar member of the device containing one or more test zones, and a second magnetic layer comprising magnetic media configured to provide an attractive force between said first magnetic layer and said second magnetic layer, wherein at least one substantially planar member comprising a hydrophilic region containing one or more test zones defined by a fluid-impermeable barrier is disposed between the first magnetic layer and the second magnetic layer.

69. An assay method comprising providing a device of any one of claims 1-68, adding a sample to the device, and detecting the presence of an analyte in said sample.
